(19) United States
(12) Patent Application Publication
(10) Pub. No.: US 2013/0115230 A1
(43) Pub. Date: May 9, 2013

(54) DELIVERY PROTEINS

(71) Applicant: Paul M. Simon, Wilmington, DE (US)

(72) Inventor: Paul M. Simon, Wilmington, DE (US)

(73) Assignee: Augmenta Biologicals, LLC, Wynnewood, PA (US)

(21) Appl. No.: 13/695,269

(22) PCT Filed: Apr. 29, 2011
(86) PCT No.: PCT/US11/34589

§ 371 Date: Jan. 7, 2013

Related U.S. Application Data
(60) Provisional application No. 61/330,075, filed on Apr. 30, 2010.

(51) Int. Cl. A61K 47/48; C07K 14/77; C07K 16/28

(52) U.S. Cl. CPC A61K 47/48561; C07K 16/28; C07K 14/77
USPC 424/178.1; 536/23.4; 530/387.3; 530/367; 435/320.1; 206/570

(57) ABSTRACT

Disclosed herein are materials and methods related to vaccines. Materials and methods for delivery of a payload, e.g., an immunogen, to the reticuloendothelial system via non-circulating lymphoid cells are provided.

### RELATED APPLICATIONS

This application claims the benefit of the filing date of U.S. Provisional Application No. 61/330,075, which was filed on Apr. 30, 2010. For the purpose of any U.S. application or patent that claims the benefit of U.S. Provisional Application No. 61/330,075, the content of that earlier filed application is hereby incorporated by reference in its entirety.

### TECHNICAL FIELD

This invention relates to materials and methods involved in immunotherapy.

### BACKGROUND

Vaccination is one of the most important medical interventions for preventing disease. The purpose of vaccination is to induce an optimal immune response that provides preventive or therapeutic benefit to the host. Vaccines typically contain one or more immunogens (IMGs) that are harmless variants or derivatives of pathogens, which act to stimulate the immune system to mount defenses against the actual pathogen. There are many types of immunogens (IMGs), ranging from attenuated or killed microorganisms, microbial extracts, whole proteins, polysaccharides, and peptides. Some IMGs are very effective inducers of the desired immune response, while others require the co-administration of non-specific immune stimulants, or adjuvants, or the coupling of the IMG to a carrier protein or microparticulate substances. Still other IMGs are inherently poor at inducing effective immune responses, despite combination with adjuvants and repeated boosts. Many of these inherently weak immunogens are involved in diseases e.g., influenza and cancer, that remain major causes of human morbidity and mortality. There is a continuing need for effective and more potent vaccines, particularly those with the capacity to stimulate a robust immune response against weakly immunogenic targets.

### SUMMARY

Disclosed herein are compositions, which we may refer to as delivery proteins, that are useful for selective delivery of a “payload”, for example, an immunogen, a toxin or an infectious agent, to the surface of a non-lymphoid cell for transport to the reticuloendothelial system (RES) for degradation and presentation to the immune system. The delivery proteins described herein can consist of, consist essentially of or comprise the recited elements. Accordingly, disclosed are compositions that consist of, consist essentially of or comprise a ligand that specifically binds to a cell surface molecule on a circulating non-lymphoid cell, wherein the cell-surface molecule is not CR1, joined to a biotin-binding protein or fragment thereof. The cell surface molecule can be any molecule that is differentially expressed on a circulating non-lymphoid cell relative to the expression level of that molecule on a cell type that is not a circulating non-lymphoid cell. In one embodiment, the cell surface molecule is selected from the group consisting of glycophorin A (CD235A), glycophorin B (CD235B), glycophorin C(CD235C), band 3 (CD233), Ter-119, blood group antigen H, blood group antigen A, blood group antigen B, Rh(D) (CD240D), Lutheran glycoprotein (CD239), Kell glycoprotein (CD258), CD41a, CD14, CD56, CD66d, CD83, CMKLR1, and BDCA-4. The ligand can be an antibody or a functional fragment thereof; the antibody can be an anti-TER-119 antibody, an anti-glycophorin A antibody, an anti-band 3 antibody, an anti-blood group antigen A antibody, an anti-blood group antigen B antibody, an anti-blood group antigen H antibody, an anti-CD41a antibody, an anti-CD14 antibody, an anti-CD56 antibody, an anti-CD66d antibody, an anti-CD83 antibody, an anti-CMKLR1 antibody, or an anti-BDCA-4 antibody. The antibody can be a single chain antibody; the single chain antibody can be a single chain variable fragment (scFv).

In one embodiment, the cell-surface molecule is on a red blood cell; the cell-surface molecule can be glycophorin A (CD235A), band 3 (CD233), TER-119, blood group antigen A, blood group antigen B, or blood group antigen H.

The biotin-binding protein can be any polypeptide that specifically binds non-covalently to biotin or a biotin mimetic, for example, streptavidin, avidin, neutravidin or an anti-biotin antibody. The biotin-binding protein can form a dimer or tetramer. The streptavidin can be a core streptavidin. In one aspect, the core streptavidin includes amino acids 249 to 374 of SEQ ID NO: 3. A biotin mimetic can be, for example, Strep-tag (IBA-go).

In one embodiment, the ligand is joined to the biotin-binding protein by a covalent bond. The ligand and the biotin-binding protein can constitute a fusion protein. The fusion protein may be a monomer. Alternatively or in addition, the fusion protein may be a dimer of two fusion proteins or a tetramer of four fusion proteins. The fusion protein can include, for example, an anti-glycophorin A antibody or antibody recognizing a glycophorin A homologue, for example, an anti-TER 119 antibody, and a core streptavidin. The fusion protein can include an amino acid sequence that is at least 80% identical to the amino acid sequence represented by SEQ ID NO: 3. The fusion protein can include an amino acid sequence that is at least 85% identical to the amino acid sequence represented by SEQ ID NO: 3. The fusion protein can include an amino acid sequence that is at least 90% identical to the amino acid sequence represented by SEQ ID NO: 3. The fusion protein can include an amino acid sequence that is at least 95% identical to the amino acid sequence represented by SEQ ID NO: 3. The fusion protein can include an amino acid sequence that is at least 98% identical to the amino acid sequence represented by SEQ ID NO: 3. The fusion protein can include the amino acid sequence represented by SEQ ID NO: 3. The fusion protein can consist of the amino acid sequence represented by SEQ ID NO: 3.

Also provided are nucleic acids comprising a nucleotide sequence encoding the fusion proteins, a vector (e.g., a vector that includes a transcriptional regulatory element operably linked the nucleotide sequence) containing the nucleic acid, and a host cell (e.g., a prokaryotic cell or a eukaryotic cell) containing the vector.

In another embodiment, the ligand and the biotin-binding protein are joined via a non-covalent bond. The non-covalent bond can be a biotin-avidin linkage.

Also provided are methods for inducing or enhancing an immune response to an immunogen in an individual, the method comprising providing an immunogen that can bind to a biotin-binding protein, for example, a biotinylated immunogen; combining the immunogen with a composition consisting of, consisting essentially of or comprising a ligand that specifically binds to a cell surface molecule on a circulating non-lymphoid cell, wherein the cell-surface molecule is not CR1, joined to a biotin-binding protein or fragment thereof, to form an immune complex; and administering an effective amount of the complex to the individual, wherein the complex induces or enhances an immune response to the immunogen. The immunogen can be influenza A M2 protein or a fragment of influenza A M2 protein. The fragment of influenza A M2 can be the ectodomain peptide M2e, for example, SEQ ID NO: 6.

Also provided are articles of manufacture that can include the delivery proteins as described herein. An article of manufacture can include, for example, a measured amount of a delivery protein, wherein the delivery protein consists essentially of a ligand that specifically binds to a cell surface molecule on a circulating non-lymphoid cell, wherein the cell-surface molecule is not CR1, joined to a biotin-binding protein or fragment thereof, and one or more items selected from the group consisting of packaging material, a package insert comprising instructions for use, a sterile fluid, and a sterile container and optionally, an adjuvant.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts a schematic of immunotargeting via an anti-TER-119 fusion protein.

FIG. 2 depicts a restriction map of the FP plasmid which encodes an anti-TER-119-ScFv-streptavidin fusion protein.

FIG. 3 depicts an SDS gel analysis of the expression and purification of an anti-TER-119-ScFv-streptavidin fusion protein. The content of lanes a-p is indicated in the table below the gel.

FIG. 4 depicts the results of an experiment analyzing the coupling of biotinylated M2e peptide to anti-TER-119-ScFv-streptavidin fusion protein.

FIG. 5 depicts the results of an experiment demonstrating the immunogenicity of M2e-FP complexes.

FIG. 6 depicts the results of an analysis of individual anti-M2e IgG titers.

FIG. 7 depicts the results of an experiment comparing the anti-M2e IgG titers obtained by intravenous, intramuscular and subcutaneous routes of injection.

FIG. 8 depicts the results of an analysis of anti-streptavidin IgG titers in animals immunized with M2e-FP complexes.

FIG. 9 depicts the DNA sequence of the pelB-Ter-streptavidin-His construct (SEQ ID NO: 1). The nucleotide sequence of the TER-119-streptavidin fusion protein encoded by pelB-Ter-streptavidin-His construct is represented by the sequence extending from nucleotide 69 to nucleotide 1196 of SEQ ID NO:1.

FIG. 10 depicts the amino acid sequence of the bacterially expressed pelB-Ter-streptavidin-His (SEQ ID NO: 2).

FIG. 11 depicts the amino acid sequence of the anti-TER-119-ScFv-streptavidin fusion protein (SEQ ID NO: 3).

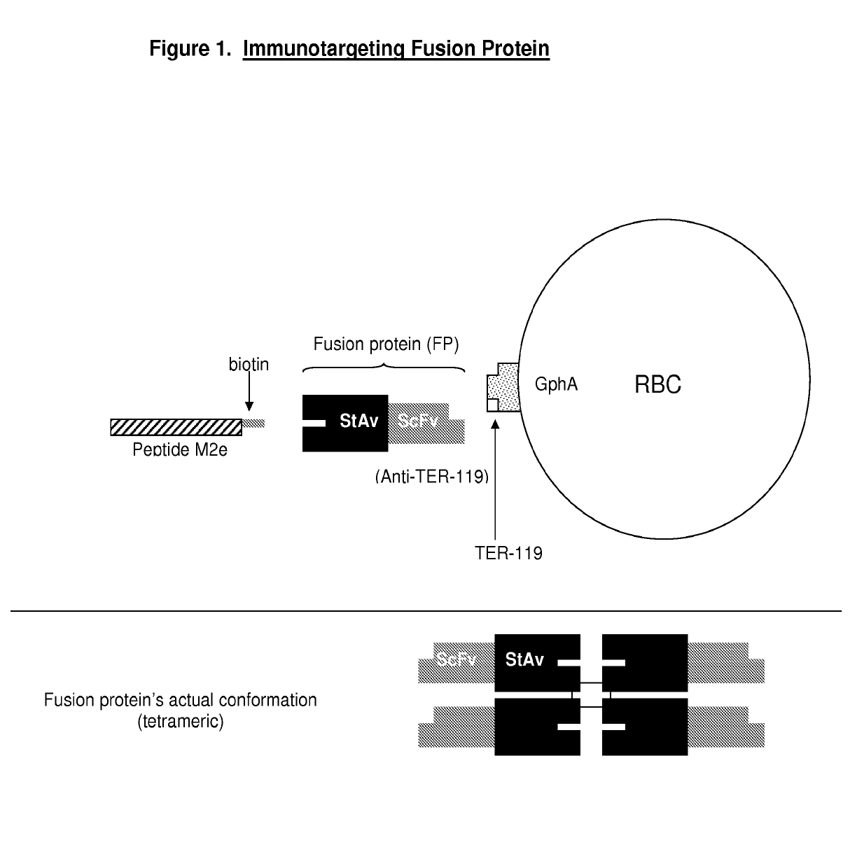

Like reference symbols in the various drawings indicate like elements.

### DETAILED DESCRIPTION

Disclosed herein are materials and methods for the specific delivery of immunogens or infectious agents to the immune system. In particular, delivery compositions are provided that specifically bind to circulating non-lymphoid cells, e.g., red blood cells, platelets, natural killer cells, monocytes, granulocytes, and plasmacytoid dendritic cells, that are cleared through the reticuloendothelial system (RES), the phagocytic system of the body that includes the fixed macrophages of tissues, liver and spleen.

The present disclosure is based, in part, on our discovery of non-naturally occurring proteins that can be used to selectively deliver a “payload”, for example, an immunogen, a toxin or an infectious agent, to the surface of a non-lymphoid cell for transport to the RES for degradation and presentation to the immune system. The proteins, which we also describe as “delivery proteins” are non-naturally occurring in the sense that they include amino acid sequences that are not normally a part of the same protein or protein complex.

Accordingly, the delivery proteins consist essentially of a ligand that specifically binds to a cell surface molecule on a circulating non-lymphoid cell and a biotin-binding protein or fragment thereof. As described further below, the cell-surface molecule can be bound by an immunoglobulin, and the ligand can therefore be a variable domain of either the immunoglobulin's light chain (V_{L}) or heavy chain (V_{H}). More specifically, the ligand can be an Fab fragment of a single chain antibody (scFv). The biotin-binding protein can be a streptavidin, an avidin, neutravidin, an antibody, e.g., an anti-biotin antibody, another biotin-binding protein or a protein that specifically binds a biotin mimetic.

The ligand and the biotin-binding polypeptide can be covalently bound to one another. For example, the ligand and the biotin binding polypeptide can be joined by a peptide bond such that the resulting delivery protein is a fusion protein. As the ligand and the biotin binding polypeptide will differ in their amino acid sequence, we may refer to them as “heterologous” polypeptides. Either polypeptide can be of a full length (i.e., it can have as many amino acid residues as its naturally-occurring counterpart) or less than full length (i.e., it can be a fragment of its naturally-occurring counterpart that retains the ability to bind its respective target. So, for example, in embodiments in which the ligand is an immunoglobulin, that immunoglobulin can be a fragment of an immunoglobulin as long as it retains the ability of the intact immunoglobulin to bind the cell-surface molecule. Similarly, the biotin-binding polypeptide can be a fragment of a naturally occurring biotin-binding polypeptide as long as it retains the ability of the native protein to bind biotin. Additional sequence can also be included. For example, the ligand and the biotin binding polypeptide may be joined by a peptide spacer. Where the delivery protein includes components from more than two polypeptides, they may be variously joined by additional spacers. The ligand and the biotin binding polypeptide can also be joined by disulphide bonds or, as noted above, be included within a protein complex in which one or more polypeptides are joined (e.g., by disulphide bonds). In other embodiments, the ligand and the biotin binding polypeptide can form the delivery protein by association within a non-covalent complex.

Amino acid sequences derived from any species, including Homo sapiens, can be used. Accordingly, the ligand or the biotin binding polypeptide can be a human polypeptide or derived from a human polypeptide (e.g., it may be a polypeptide that differs from a naturally occurring human sequence due to the introduction of one or more mutations). Where the ligand or the biotin binding polypeptide is an immunoglobulin or a fragment of an immunoglobulin, the immunoglobulin can be a human or “humanized” immunoglobulin. Proteins from other species, including rodents and domestic animals, can be used (particularly where veterinary applications are indicated; the polypeptides incorporated into the delivery protein can be from the same genus or species of animal being treated).

Various types of polypeptides can be used as either the ligand or the biotin binding polypeptide, and the ligand and the biotin binding polypeptide can be of the same type. For example, the delivery protein can be a bispecific diabody, which includes V_{H} and V_{L} of an immunoglobulin that binds a first target and V_{H} and V_{L} of an immunoglobulin that binds a second target. Thus, the ligand and the biotin binding polypeptide can be of the same type; both can be a variable region of an immunoglobulin chain.

More generally, the ligand and/or the biotin binding polypeptide can be an immunoglobulin chain or an antigen-binding fragment thereof. For example, the delivery protein can include an immunoglobulin (i.e., a protein complex) or an antigen-binding fragment thereof in which the ligand and/or the biotin binding polypeptide includes at least three complementarity determining regions. The immunoglobulin can be a member of the G or M class (i.e., an IgG or IgM).

Accordingly, the delivery proteins consist essentially of a ligand that specifically binds to a cell surface molecule on a circulating non-lymphoid cell and a biotin-binding protein or fragment thereof. The phrase “consisting essentially of” indicates that the delivery proteins necessarily include the listed elements, e.g., a ligand that specifically binds to a cell surface molecule on a circulating non-lymphoid cell and a biotin-binding protein or fragment thereof, and are open to unlisted elements that do not materially affect the basic and novel properties of the delivery protein. Thus, the unlisted elements are limited to other elements that are optional and may or may not be present. Such unlisted elements, if present, would not adversely affect either the cell surface binding or the biotin binding functions of the delivery protein. Such elements could include, for example, tag sequences for facilitating purification, localization signals, and labels for detection. The unlisted elements within the scope of “consisting essentially of” would not include the payload described below.

The delivery proteins can be used to deliver a payload, e.g., an immunogen, a toxin or an infectious agent, to the surface of a red blood cell for transport to the RES for degradation and presentation to the immune system. The payload can take many forms and will vary depending upon the nature of the target against which an immune response is desired, the structure of the immunogen, and the relative immunogenicity of the immunogen, but generally, the delivery proteins of the claims do not encompass the payload itself. So, for example, a delivery protein can be a fusion protein that includes an antibody or other protein, e.g., a blood group specific lectin, that selectively binds to a cell surface antigen on a red blood cell, fused in frame to an biotin-binding protein. Alternatively, a delivery protein can be an antibody or other protein, e.g., a blood group specific lectin, that selectively binds to a cell surface antigen on a red blood cell, bound to a biotin binding protein. The antibody or other protein and the biotin-binding protein can be joined covalently or non-covalently, but regardless of the way in which they are joined, such delivery proteins do not include the payload. Thus, the delivery protein does not include a payload, for example, an immunogen, a toxin or an infectious agent.

### Compositions

### Circulating Non-Lymphoid Cells and Cell Surface Molecules

As used herein, a circulating non-lymphoid cell can be any cell that a) circulates through the body of an animal, e.g., a mammal, a fish or a bird, in the blood and/or lymph system; and b) is not a B lymphocyte or a T lymphocyte. Thus, circulating non-lymphoid cells include erythrocytes, i.e., red blood cells; platelets; natural killer cells; monocytes; granulocytes, i.e., neutrophils, eosinophils, and basophils; and plasmacytoid dendritic cells.

B and T lymphocytes are ultimately derived from hematopoietic stem cells and perform the principal functions of the immune system. T lymphocytes mature through the thymus and are generally identified by their expression of CD3 (which is associated with the T cell receptor) and either CD4 or CD8. CD8-expressing (or CD8+) T cells are principally involved with direct cell killing, or cytotoxicity. CD4+ T cells are primarily regulatory cells which stimulate and suppress immune responses, as needed. B lymphocytes are characterized by their expression of CD19 or CD20, among other surface markers, and they are responsible for antibody production. B cells are also effective antigen presenting cells.

Erythrocytes, also known as red blood cells, are the most abundant cell type in mammalian blood. They are small disc-shaped, anucleated, biconcave cells whose primary function is to carry oxygen and carbon dioxide to and from the tissues. Red blood cells express a distinctive complement of cell surface markers, including the human blood group antigens, glycophorins, band 3 and the Lewis antigens.

Platelets are derived from megakaryocytes, they are centrally involved in blood clotting, and can be identified by their surface expression of CD41a (or gpIIb/IIIa). Natural killer cells, also referred to as large granular lymphocytes, are derived from the bone marrow and do not express T-cell antigen receptors (TCR), the pan-T marker CD3 or surface immunoglobulins (Ig) B cell receptor, but typically express the surface markers CD16 (FcγRIII) and CD56. Monocytes are derived from myeloid stem cells and are found primarily in the circulation. They are competent phagocytes. Upon their binding of pathogens and/or stimulation by various cytokines, monocytes mature into macrophages, which are even more avid phagocytes and producers of many cytokines, degradative enzymes and other molecules that mediate inflammatory reactions. Macrophages are generally found bound to vascular endothelium or within various tissues. Monocytes (and macrophages) are characterized by the surface expression of CD14, among other markers.

Granulocytes are also derived from myeloid stem cells and are characterized by the presence of abundant granules in their cytoplasm; different classes of granulocytes, e.g., eosinophils, basophils and neutrophils, are distinguished by their ability to stain with eosin, basophilic dyes or neither, respectively. Eosinophils are involved in defense against parasitic pathogens and allergens; basophils are also involved in allergic reactions. Neutrophils are early and aggressive phagocytes at the site of infections and release products that induce inflammatory reactions.

Plasmacytoid dendritic cells (pDC) are distinct from myeloid dendritic cells. Both are found in the circulation and in tissues. pDC are an important link between the innate and adaptive immune responses, in particular in mounting anti-viral immune responses. They produce abundant interferons and can be identified by the surface marker BDCA-2 (CD303).

The circulating non-lymphoid cells can be derived from any animal, e.g., humans, non-human primates, cattle, horses, pigs, sheep, goats, deer, elk, dogs, cats, mustelids, rabbits, guinea pigs, hamsters, rats, mice, fish, for example salmon, carp and tilapia, or birds, for example, chickens, turkeys, ducks or geese.

Any cell-surface molecule, provided that it is not CR1 (also known as erythrocyte complement receptor 1, CD35, C3b/C4b receptor and immune adherence receptor), that is differentially expressed on circulating non-lymphoid cells relative to the levels of the same molecule on a cell type that is not a circulating non-lymphoid cell is a suitable target for the ligand. The cell-surface molecule can be a polypeptide, a carbohydrate, or a glycolipid. Full-length molecules, epitopes, analogs, mutants, and functional fragments thereof are encompassed by this definition. A “functional fragment” of a molecule is a fragment of the molecule that is smaller (shorter where the molecule is a polypeptide) than the molecule per se but has at least 10% (e.g., 10%, 20%, 30%, 40%, 50%, 60%, 70%, 80%, 90%, 95%, 98%, 99%, 99.5%, 100% or even more) of the ligand-binding activity of the molecule per se.

Typically, a cell-surface molecule on a circulating non-lymphoid cell can be classified as being differentially expressed if the molecule is present at a level that is greater than the average level observed in cells that are not circulating non-lymphoid cells if the expression levels differ by at least 50% (e.g., 50, 100, 200, 300% or more). Any method can be used to determine whether or not a specific gene product is expressed at a level that is greater or less than the average level of expression observed in control cells. The level of expression of a cell surface polypeptide can be measured using any method such as immuno-based assays (e.g., immunofluorescence, flow cytometry, ELISA), western blotting, or polyacrylamide gel electrophoresis with silver staining Levels of particular carbohydrates or lipids can be measured by immunodetection e.g., ELISA, flow cytometry, or immunostaining using fluorochrome- or radioisotope-labeled antibodies or lectins. In some embodiments, the level of expression from a particular gene can be measured by assessing the level of mRNA expression from the gene. Levels of mRNA expression can be evaluated using, without limitation, northern blotting, slot blotting, quantitative reverse transcriptase polymerase chain reaction (RT-PCR), or chip hybridization techniques. Such methods can be used to determine simultaneously the relative expression levels of multiple mRNAs.

Examples of targets on red blood cells include, without limitation, glycophorin A (CD235A), glycophorin B (CD235B), glycophorin C(CD235C), band 3 (CD233), TER-119 (Kina et al., Br. J. Hematol. 109: 280-7, 2000), the ABO blood group antigens, e.g., blood group antigen A, blood group antigen B, blood group antigen H, and phosphatidyl serine (Hematology: Basic Principles and Practice, R. Hoffman, 2005, 4^{th} ed. New York: Churchill-Livingstone). One useful red blood cell target is TER-119, a region selectively bound by the antibody, anti-TER-119 and which is associated with the extracellular domain of glycophorin A. Suitable platelet targets includes gpIIb/IIIa (CD41a), CD42d, CD61, CD62P (P-selectin) and CD151. Natural killer cell targets include, for example, CD56 (NCAM, Leu-19, HNK1). Monocyte and granulocyte targets include, for example, CD14 (LPS-receptor) and CD66d (CGM1, a member of the CEA family), respectively. Plasmacytoid dendritic cell targets include, for example, CMKLR1 (serpentine chemokine-like receptor 1), BDCA-2 (CD303) and BDCA-4 (CD304) (Kuby Immunology, J. Kuby et al., edt., 2002, 5^{th} edition, W.H.Freeman & Co.).

### Ligands

The term “ligand” as used herein refers to a molecule capable of binding a specific cell surface molecule on a circulating non-lymphoid cell. A ligand can be a polypeptide, a carbohydrate, glycolipid or biomimetic of a polypeptide, carbohydrate or glycolipid, as long as the ligand binds specifically to a cell surface molecule that is differentially expressed on a circulating non-lymphoid cell and that is not CR1.

As defined herein, ligands are defined to be “specifically binding” if: 1) they exhibit a threshold level of binding activity, and/or 2) they do not significantly cross-react with related target molecules. The binding affinity of a ligand can be readily determined by one of ordinary skill in the art, for example, by Scatchard analysis (Scatchard, Ann. NY Acad. Sci. 51: 660-672, 1949). For example, a ligand disclosed herein can bind to its target with at least 1.5-fold, 2-fold, 5-fold, 10-fold, 100-fold, 103-fold, 104-fold, 105-fold, 106-fold or greater affinity for the target than for a closely related or unrelated polypeptide. A ligand can bind its target with high affinity (10⁻⁴M or less, 10⁻⁷M or less, 10⁻⁹M or less, or with subnanomolar affinity (0.9, 0.8, 0.7, 0.6, 0.5, 0.4, 0.3, 0.2, 0.1 nM or even less). Ligands can also be described or specified in terms of their binding affinity to a target, for example, binding affinities include those with a Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹M, 10⁻¹¹M, 5×10⁻¹²M, 10⁻¹²M, 5×10⁻¹³ M, 10⁻¹³ M, 5×10⁻¹⁴ M, 10⁻¹⁴ M, 5×10⁻¹⁵ M, or 10⁻¹⁵ M, or less.

In some embodiments, the ligands disclosed herein do not bind to known related molecules. In other embodiments, the ligands disclosed herein can bind to orthologs, homologs, paralogs or variants, or combinations and subcombinations thereof, of their targets.

As defined herein, a ligand that specifically binds to circulating non-lymphoid cells is a ligand that binds to circulating non-lymphoid cells and does not significantly bind to lymphoid cells. For example, a delivery protein that includes a ligand that specifically binds to red blood cells will bind to red blood cells and not significantly bind to other circulating cells, e.g., lymphocytes, platelets, natural killer cells, monocytes, granulocytes, endothelial cells, fibroblasts or dendritic cells. In another example, a delivery protein that includes a ligand that specifically binds to platelets will bind to platelets and not significantly bind to other circulating cells, e.g., lymphoid cells, red blood cells, natural killer cells, monocytes, granulocytes, endothelial cells, fibroblasts or dendritic cells.

Ligands may be screened against known related target polypeptides to isolate a ligand that specifically binds the target. For example, a ligand specific to a target will flow through an affinity chromatography column comprising other closely related target molecules adhered to insoluble matrix under appropriate buffer conditions. Such screening allows isolation of ligands non-crossreactive to closely related targets (Antibodies: A Laboratory Manual, Harlow and Lane (eds.), Cold Spring Harbor Laboratory Press, 1988; Current Protocols in Immunology, Cooligan et al. (eds.), National Institutes of Health, John Wiley and Sons, Inc., 1995). Screening and isolation of specific antibodies is well known in the art (see, Fundamental Immunology, W. Paul (ed.), Raven Press, 1993; Getzoff et al., Adv. in Immunol. 43: 1-98, 1988; Monoclonal Antibodies: Principles and Practice, Goding, J. W. (ed.), Academic Press Ltd., 1996; Benjamin et al., Ann. Rev. Immunol. 2: 67-101, 1984). Representative examples of such assays include: concurrent immunoelectrophoresis, radioimmunoassay (RIA), radioimmunoprecipitation, flow cytometry, FACS, enzyme-linked immunosorbent assay (ELISA), dot blot or western blot assay, inhibition or competition assay, and sandwich assay.

A ligand can be a polypeptide, provided that it does not bind CR1. We tend to use the term “protein” to refer to longer or larger amino acid polymers, and we tend to use the term “polypeptide” to refer to shorter sequences or to a chain of amino acid residues within a larger molecule (e.g., within a fusion protein) or complex. Both terms, however, are meant to describe an entity of two or more subunit amino acids, amino acid analogs, or other peptidomimetics, regardless of post-translational modification (e.g., amidation, phosphorylation or glycosylation). The subunits can be linked by peptide bonds or other bonds such as, for example, dicysteine, ester or ether bonds. The terms “amino acid” and “amino acid residue” refer to natural and/or unnatural or synthetic amino acids, which may be D- or L-form optical isomers. Full-length proteins, analogs, mutants, and fragments thereof are encompassed by this definition.

The amino acid sequence of the ligands disclosed herein can be identical to the wild-type sequences of appropriate components. Alternatively, any of the components can contain mutations such as deletions, additions, or substitutions. All that is required is that the variant ligand have at least 5% (e.g., 10%, 20%, 30%, 40%, 50%, 60%, 70%, 80%, 90%, 95%, 99%, 100%, or even more) of the ability of the ligand containing only wild-type sequences to specifically bind the target on the circulating non-lymphoid cell. Substitutions will preferably be conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Any method can be used to make a polypeptide including, for example, expression by prokaryotic systems, expression by eukaryotic systems, and chemical synthesis techniques. Any method can be used to purify a polypeptide including, without limitation, fractionation, centrifugation, and chromatography, e.g., gel filtration, ion exchange chromatography, reverse-phase HPLC and immunoaffinity purification.

A polypeptide ligand within the delivery protein can be, or can be a part of, an immunoglobulin. The immunoglobulins can assume various configurations and encompass proteins consisting of one or more polypeptides substantially encoded by immunoglobulin genes. We may use the term “immunoglobulin” synonymously with “antibody.”

An immunoglobulin can be a tetramer (e.g., an antibody having two heavy chains and two light chains) or a single-chain immunoglobulin, and any of the polypeptides in the tetramer or the single polypeptide of the single chain antibody may be used as the ligand and/or the biotin binding polypeptide of the present delivery proteins. Accordingly, the ligand can be one of the two heavy chains or heavy chain variable regions or one of the two light chains or light chain variable regions. The V_{H} and V_{L} regions are further subdivided into regions of hypervariability, termed “complementarity determining regions” (CDRs), interspersed with the more conserved framework regions (FRs). The extent of the FRs and CDRs has been defined (see, Kabat, E. A., et al. Sequences of Proteins of Immunological Interest, Fifth Edition, U.S. Department of Health and Human Services, NIH Publication No. 91-3242, 1991, and Chothia, et al., J. Mol. Biol. 196:901-917, 1987, which are incorporated herein by reference).

The V_{H} or V_{L} chain of an immunoglobulin can further include all or part of a heavy or light chain constant region. For example, the present ligand polypeptide can be within an immunoglobulin tetramer of two heavy immunoglobulin chains and two light immunoglobulin chains, wherein the heavy and light immunoglobulin chains are inter-connected by, e.g., disulfide bonds. The heavy chain constant region includes three domains: CH1, CH2 and CH3. The light chain constant region is comprised of one domain: CL. The variable regions of the heavy and light chains contain a binding domain that interacts with an antigen. The polypeptides may be those of intact immunoglobulins of types IgA, IgG, IgE, IgD, IgM (as well as subtypes thereof (e.g., IgG₁, IgG₂, IgG₃, and IgG₄)), and the light chains of the immunoglobulin may be of types kappa or lambda. The recognized human immunoglobulin genes include the kappa, lambda, alpha (IgA₁ and IgA₂), gamma (IgG₁, IgG₂, IgG₃, IgG₄), delta, epsilon, and mu constant region genes, as well as the myriad immunoglobulin variable region genes.

Polypeptides within the delivery proteins of the claims may include CDRs from a human or non-human source. The framework of the immunoglobulin can be human, humanized, or non-human (e.g., a murine framework modified to decrease antigenicity in humans), or a synthetic framework (e.g., a consensus sequence). Humanized immunoglobulins are those in which the framework residues correspond to human germline sequences and the CDRs result from V(D)J recombination and somatic mutations. However, humanized immunoglobulins may also comprise amino acid residues not encoded in human germline immunoglobulin nucleic acid sequences (e.g., mutations introduced by random or site-specific mutagenesis ex vivo). It has been demonstrated that in vivo somatic mutation of human variable genes results in mutation of framework residues (see Nat. Immunol. 2:537, 2001). Such an antibody would be termed “human” given its source, despite the framework mutations. Mouse antibody variable domains also contain somatic mutations in framework residues (See Sem. Immunol. 8:159, 1996). Consequently, transgenic mice containing the human Ig locus produce immunoglobulins that are commonly referred to as “fully human,” even though they possess an average of 4.5 framework mutations (Nature Genet. 15:146-56, 1997). Accepted usage therefore indicates that an antibody variable domain gene based on germline sequence but possessing framework mutations introduced by, for example, an in vivo somatic mutational process is termed “human.” As noted above, the present delivery proteins encompass those that specifically bind a cell surface antigen and a biotin-binding protein even where those proteins include mutations (e.g., mutations within the FR) and fragments or other variants thereof (e.g., single chain antibodies that include the V_{L} and V_{H} of a multimeric human antibody).

The term “antigen-binding portion” of an immunoglobulin or antibody (or simply “antibody portion,” or “portion”), as used herein, refers to a portion of an immunoglobulin that specifically binds to a cellular target, e.g., a cell surface antigen. An antigen-binding portion of an immunoglobulin is therefore a molecule in which one or more immunoglobulin chains are not full length, but which specifically binds to a cellular target. Examples of antigen-binding portions or fragments that can be used in the present proteins include: (i) an Fab fragment, a monovalent fragment consisting of the V_{L}, V_{H}, CL and CH1 domains; (ii) an F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., Nature 341:544-546, 1989), which consists of a V_{H} domain; and (vi) an isolated complementarity determining region (CDR) having sufficient framework to specifically bind, e.g., an antigen binding portion of a variable region. An antigen-binding portion of a light chain variable region and an antigen binding portion of a heavy chain variable region, e.g., the two domains of the Fv fragment, V_{L} and V_{H}, can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423-426, 1988; and Huston et al., Proc. Natl. Acad. Sci. USA 85:5879-5883, 1988). Such single chain antibodies are also intended to be encompassed within the term “antigen-binding portion” of an antibody.

These antibody portions are obtained using conventional techniques known to those with skill in the art, and the portions are screened for utility in the same manner as are intact antibodies. An Fab fragment can result from cleavage of a tetrameric antibody with papain; Fab′ and F(ab′)2 fragments can be generated by cleavage with pepsin.

In summary, single chain immunoglobulins, and chimeric, humanized or CDR-grafted immunoglobulins, including those having polypeptides derived from different species, can be incorporated into the delivery proteins.

The various portions of these immunoglobulins can be joined together chemically by conventional techniques, or can be prepared as contiguous polypeptides using genetic engineering techniques. For example, nucleic acids encoding a chimeric or humanized chain can be expressed to produce a contiguous polypeptide. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; and Winter, European Patent No. 0,239,400 B1. See also, Newman et al., BioTechnology 10:1455-1460, 1992, regarding CDR-graft antibody, and Ladner et al., U.S. Pat. No. 4,946,778 and Bird, R. E. et al., Science 242: 423-426, 1988 regarding single chain antibodies.

Nucleic acid (e.g., DNA) sequences coding for any of the polypeptides within the present delivery proteins are also within the scope of the present invention as are methods of making the delivery proteins. For example, variable regions can be constructed using PCR mutagenesis methods to alter DNA sequences encoding an immunoglobulin chain, e.g., using methods employed to generate humanized immunoglobulins (see e.g., Kanunan, et al., Nucl. Acids Res. 17: 5404, 1989; Sato, et al., Cancer Research 53: 851-856, 1993; Daugherty, et al., Nucleic Acids Res. 19(9): 2471-2476, 1991; and Lewis and Crowe, Gene 101: 297-302, 1991). Using these or other suitable methods, variants can also be readily produced. In one embodiment, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993)).

Other suitable methods of producing or isolating immunoglobulins that specifically recognize a cellular target include, for example, methods that rely upon immunization of transgenic animals (e.g., mice) capable of producing a full repertoire of human antibodies (see e.g., Jakobovits et al., Proc. Natl. Acad. Sci. USA 90: 2551-2555, 1993; Jakobovits et al., Nature 362: 255-258, 1993; Lonberg et al., U.S. Pat. No. 5,545,806; Surani et al., U.S. Pat. No. 5,545,807). These U.S. patents are incorporated by reference herein.

The binding affinities of an immunoglobulin or of any of the other types of binding entities useful in the present delivery proteins can be readily determined by one of ordinary skill in the art, for example, by Scatchard analysis (Scatchard, Ann. NY Acad. Sci. 51:660-672, 1949). For example, the immunoglobulins can bind with high affinity of 10⁻⁴M or less, 10⁻⁷M or less, 10⁻⁹M or less or with subnanomolar affinity (0.9, 0.8, 0.7, 0.6, 0.5, 0.4, 0.3, 0.2, 0.1 nM or even less). Immunoglobulins may also be described or specified in terms of their binding affinity for their specific cellular targets.

The immunoglobulins may be modified to reduce or abolish glycosylation. An immunoglobulin that lacks glycosylation may be an immunoglobulin that is not glycosylated at all; that is not fully glycosylated; or that is atypically glycosylated (i.e., the glycosylation pattern for the mutant differs from the glycosylation pattern of the corresponding wild type immunoglobulin). The IgG polypeptides include one or more (e.g., 1, 2, or 3 or more) mutations that attenuate glycosylation, i.e., mutations that result in the an IgG CH2 domain that lacks glycosylation, or is not fully glycosylated or is atypicially glycosylated.

Methods for producing antibodies are well know to those in the art; Antibodies can be purified by chromatographic methods known to those of skill in the art, including ion exchange and gel filtration chromatography (for example, Caine et al., Protein Expr. Purif. (1996) 8(2):159-166). Alternatively or in addition, antibodies can be purchased from commercial sources, for example, Invitrogen (Carlsbad, Calif.); MP Biomedicals (Solon, Ohio); Nventa Biopharmaceuticals (San Diego, Calif.) (formerly Stressgen); Serologicals Corp. (Norcross, Ga.).

The antibody can be, for example, an antibody that recognizes a target, provided that the target is not CR1, on red blood cells, including for example, without limitation, glycophorin A (CD235A), glycophorin B, glycophorin C, band 3 (CD233), TER-119, blood group antigen A, blood group antigen B, and blood group antigen H. One useful antibody is anti-TER-119, which specifically binds to TER-119, a region corresponding associated with the extracellular domain of glycophorin A. Other suitable antibodies include, without limitation, antibodies that recognize targets on platelets, e.g., gpIIb/IIIa (CD41a), CD42d, CD61, CD62P(P-selectin) and CD151; monocytes, e.g., CD14; NK cells, e.g., CD56; granulocytes, e.g., CD66d; and plasmacytoid dendritic cells, e.g., CMKLR1, BDCA-2 (CD303) and BDCA-4 (CD304).

A ligand can also be a polypeptide that is not an immunoglobulin. One non-immunoglobulin type ligand can be the erythrocyte-binding antigen 175 (EBA-175) of Plasmodium falciparum, which specifically binds the red blood cell surface protein band 3, or a fragment of EBA-175 that binds to a red blood cell, for example EBA-175 peptide including amino acids 1085-96 (SEQ ID NO:4). A polypeptide ligand can also be a lectin, e.g. a glycoprotein that recognizes blood group antigen A, blood group antigen B or blood group antigen H.

A ligand can also be a peptidomimetic, a small protein-like chain containing non-peptidic structural elements that is capable of mimicking or antagonizing the biological action(s) of a natural parent peptide. Peptidomimetic compounds are synthetic, non-peptide compounds having a three-dimensional conformation (i.e., a “peptide motif”) that is substantially the same as the three-dimensional conformation of a selected peptide. The peptide motif provides the peptidomimetic compound with the ability to bind the ligand in a manner qualitatively identical to that of the parent peptide from which the peptidomimetic was derived. Peptidomimetic compounds can have additional characteristics that enhance their therapeutic utility, such as increased prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics. Any peptidomimetic that binds specifically and selectively to a cell surface molecule that is differentially expressed on a circulating non-lymphoid cell can be used. Examples of useful peptidomimetics include those that mimic the ability of antibodies that recognize, for example, glycophorin A (CD235A), glycophorin B, glycophorin C, band 3 (CD233), TER-119, blood group antigen A, blood group antigen B, and blood group antigen H, gpIIb/IIIa (CD41a), CD42d, CD61, CD62P (P-selectin), CD151, CD14, CD56, CD66d, CMKLR1 and BDCA-2.

The polypeptide can include post-translational modifications, i.e., chemical modification of the polypeptide after its synthesis. Chemical modifications can be naturally occurring modifications made in vivo following translation of the mRNA encoding the polypeptide or synthetic modifications made in vitro. A polypeptide can include one or more post-translational modifications, in any combination of naturally occurring, i.e., in vivo, and synthetic modifications made in vitro. Examples of post-translational modifications include, but are not limited to, biotinylation, e.g., acylation of lysine or other reactive amino acid residues with a biotin molecule or a biotin biomimic; glycosylation, e.g., addition of a glycosyl group to either asparagines, hydroxylysine, serine or threonine residues to generate a glycoprotein or glycopeptide; acetylation, e.g., the addition of an acetyl group, typically at the N-terminus of a polypeptide; alkylation, e.g., the addition of an alkyl group; isoprenylation, e.g., the addition of an isoprenoid group; lipoylation, e.g. attachment of a lipoate moeity; phosphorylation, e.g., addition of a phosphate group to serine, tyrosine, threonine or histidine.

An exemplary post-translational modification can be biotinylation. Biotin, also known as vitamin H or B7, is a water-soluble B-complex vitamin that binds avidin or streptavidin with very high affinity (10⁻¹⁵M). Both egg avidin and bacterial streptavidin have four biotin-binding sites and thus can serve to couple several biotinylated ligands or to couple biotinylated ligand(s) to other biotinylated molecules (e.g., IMGs). Polypeptides can be covalently linked to one or more biotin molecules through reactive groups including primary amines, e.g. lysine and N-terminus), carboxyl groups found on aspartic- and glutamic-acid residues and at the C-terminus, sulfhydryl groups, or carbohydrate modifications on glycoproteins. Methods for derivatizing polypeptides with biotin are well known in the art and there are many commercial sources for such reagents (e.g. Pierce, Sigma-Aldrich). Any method of biotinylation that preserves the ability of the ligand to bind to its target on the non-circulating lymphoid cell can be used. For example, desirable biotinlyation methods would modify residues in the scFv portion of the antibody without compromising the immunoreactivity of the antibody fragment. Polypeptides derivatized with biotin can then be linked to a biotin-binding polypeptide, e.g., through an avidin or streptavidin molecule.

A post-translational modification can be glycosylation, i.e., the addition of saccharides. Glycosylation is typically classified based on the amino acid through which the saccharide linkage occurs and can include: N-linked glycosylation to the amide nitrogen of asparagines side chains, O-linked glycosylation to the hydroxyloxygen of serine and threonine side chains, and C-mannosylation.

A ligand can also be a carbohydrate or glycolipid. Examples of carbohydrate and glycolipid ligands, include, without limitation, bacterial lipopolysaccharide (LPS) or a fragment of it, microbial products bound by Toll-like receptors (TLRs), bacterial diacyl and triacyl lipopeptides and lipoteichoic acid from bacteria, and zymosan from yeast cell walls.

A ligand can also be a nucleic acid. Examples of nucleic acids include, without limitation, single- and double-stranded RNA from viruses, and CpG DNA from bacteria or viruses.

### Biotin-Binding Proteins

The delivery proteins of the claims include a biotin-binding protein, i.e., a protein that specifically binds non-covalently to biotin (also known as vitamin B7, vitamin H or coenzyme R) or a biotin derivative, for example, iminobiotin, biotin sulfoxide, bisnobiotin, chloroacetyl biotin, biocytin and 2′-hydroxyazobenzene-4′-carboxylic acid (HABA). Exemplary biotin-binding proteins include, without limitation, avidin, streptavidin, neutravidin, which we collectively refer to as (strept)avidin, and anti-biotin antibodies. The delivery proteins include polypeptides derived from a biotin-binding protein, which may be either a mature biotin-binding protein and/or a biologically active variant thereof. A polypeptide that has an amino acid sequence that is identical to a portion of a biotin-binding protein sequence and that functions (e.g., for one or more of the purposes described herein) is a fragment of a biotin-binding protein. A polypeptide that has a sequence that differs to a certain limited extent from a sequence that is found in a naturally occurring biotin-binding protein and that retains the ability to function (e.g., retains sufficient activity to be used for one or more of the purposes described herein) is a biologically active variant of a biotin-binding protein. We tend to use the term “biotin-binding protein” to refer to full-length, naturally-occurring biotin-binding proteins, and we tend to use the terms “polypeptide” and “peptide” when referring to fragments thereof (i.e., to fragments of biotin-binding proteins) and biologically active variants thereof.

The (strept)avidins are structurally and functionally analogous polypeptides with an extremely high affinity for biotin (K_{d} of about 10⁻¹⁴ to 10⁻¹⁶ M). They are also stable against heat, extremes of pH and to activity of certain proteolytic enzymes. Wild-type (strept)avidins are tetrameric proteins, with four biotin-binding sites.

Avidin is produced in the oviducts of birds, reptiles and amphibians and deposited in the whites of their eggs. The avidin tetramer is composed of four identical subunits, each of which can bind to biotin with a high degree of affinity and specificity. Tetrameric avidin is between 66-69 kDa in size, with about 10% percent of the molecular weight derived from carbohydrate structures of four to five mannose and three N-acetylglucosamine residues. The carbohydrate moieties of avidin contain at least three unique oligosaccharide structural types. Avidin is highly cationic with an isoelectric point of about 10.5. Representative avidin sequences include GenBank accession numbers: CAC34569.1 (public GI:13397826) and AAN38297.1 (public GI:23507705). The delivery proteins of the claims can include mature avidin, or a sequence that is at least 50%, 60%, 70%, 80%, 85%, 90%, 95%, or 99% identical to the sequence identified by GenBank accession numbers: CAC34569.1 (public GI:13397826) and AAN38297.1 (public GI:23507705).

Streptavidin is a nonglycosylated 52,800-dalton protein with a near-neutral isoelectric point, originally purified from the bacterium Streptomyces avidinii. The secondary structure of a streptavidin monomer is composed of eight antiparallel β-strands, which fold to give an antiparallel β-barrel tertiary structure. A biotin binding-site is located at one end of each β-barrel. Four identical streptavidin monomers (i.e., four identical β-barrels) associate to form streptavidin's tetrameric quaternary structure. The biotin binding-site in each barrel consists of residues from the interior of the barrel, together with a conserved Trp120 from the neighboring subunit. Each subunit thus contributes to the binding site on the neighboring subunit, and so the tetramer can also be considered a dimer of functional dimers. Representative streptavidin sequences include GenBank accession numbers CAA00084.1 (public GI:14606); and ACL82594.1 (public GI:220900811). The delivery proteins of the claims can include mature streptavidin, or a sequence that is at least 50%, 60%, 70%, 80%, 85%, 90%, 95%, or 99% identical to the sequence identified by Genbank accession numbers CAA00084.1 (public GI:14606); and ACL82594.1 (public GI:220900811). The N and C termini of the 159 residue full-length protein can be deleted to produce a shorter ‘core’ streptavidin, typically composed of residues 13-139. Other examples of truncated versions of streptavidin, i.e., core streptavidin, include sequences with residues 14-138, 13-138 and 14-139 (Pähler A, Hendrickson W A, Kolks M A, Argara{tilde under (n)}a C E, Cantor C R, 1987, Characterization and crystallization of core streptavidin. J. Biol. Chem. 262:13933-7). So for example, the sequence from amino acids 14-140, 13-139, 14-138, 13-138, 14-139 of CAA00084.1 (public GI:14606) are each representative core streptavins.

NeutrAvidin protein is a deglycosylated avidin, with a mass of approximately 60,000 daltons and a near-neutral pI (6.3).

A (strept)avidin may be a monomer, dimer, or tetramer, capable of forming monovalent, divalent, or tetravalent compositions, respectively. A (strept)avidin can be a truncated form of a mature polypeptide, e.g., a core streptavidin or a single chain (strept)avidin in which all four biotin-binding domains are contained in a single polypeptide chain. Alternatively or in addition, a (strept)avidin can comprise one or more mutations relative to the wild-type sequence. A (strept)avidin can include mutations that confer pH-adjustable biotin binding to control the valency, e.g., monomeric, dimeric, or tetrameric binding to biotin. Exemplary pH-sensitive point mutants of avidin include Y33H as well as substitutions of histidine for Met96, Val115, and Ile117, optionally with histidine replacement at Trp110. Approaches for controlling biotin-streptavidin binding are described in Laitinen, O. H. (2007) Trends in Biotechnology 25 (6): 269-277 and Nordlund, H. R. (2003) FEBS Letters 555: 449-454, the contents of both of which are incorporated herein by reference.

A biotin-binding polypeptide can also be an anti-biotin antibody. Such antibodies can be a tetramer (e.g., an antibody having two heavy chains and two light chains) or a single-chain immunoglobulin, and any of the polypeptides in the tetramer or the single polypeptide of the single chain antibody as described above. Representative anti-biotin antibodies include those cited in Papasarantos I, Klimentzou P, Koutrafouri V, Anagnostouli M, Zikos C, Paravatou-Petsotas M, Livaniou E. (2010) Appl Biochem Biotechnol. 162:221-32 and Coene E D, Shaw M K, Vaux D J. (2008) Methods Mol. Biol. 418:157-70, Bagci, H., Kohen, F., Kuscuoglu, U., Bayer, E. A. and Wilchek, M. (1993) FEBS Lett. 322: 47-50, each of which is herein incorporated by reference. A representative anti-biotin immunoglobulin heavy chain is the sequence with the GenBank accession AAB26438.1 (public GI:299966).

### Delivery Protein Formats

The ligand and the biotin-binding protein can be joined in any manner, so long as the ligand and the biotin binding protein retain the ability to function (e.g., retain sufficient activity to be used for one or more of the purposes described herein). Thus, the linkage between the ligand and the biotin-binding protein can be covalent or non-covalent or even a mixture of covalent and non-covalent linkages. A linkage can be via any reagent, molecule or macromolecule that connects the ligand and the biotin-binding protein such that a) the delivery protein is stable under physiological conditions; b) the connection between the linkage and the ligand does not alter the ability of the ligand to bind to its target on the surface of a circulating non-lymphoid cell; and c) the connection between the linkage and the biotin-binding protein does not substantially affect the capacity of the biotin-binding protein to bind biotin with high affinity.

### Fusion Proteins.

The ligand and the biotin-binding polypeptide can be joined by a peptide bond. That is, the ligand and the biotin-binding polypeptide can be a fusion polypeptide comprising one or more amino acid segments from the ligand and one or more amino acid segments from the biotin-binding polypeptide. The term “amino acid segment” as used herein refers to a contiguous stretch of amino acids within a polypeptide. For example, the amino acid residues 30 to 40 within a 100 amino acid polypeptide would be considered an amino acid segment. An amino acid segment can be a length greater than eight amino acid residues (e.g., greater than about nine, ten, 15, 20, 25, 30, 40, 50, 75, 100, 150, 200, 500, 1000, or more amino acid residues). In some embodiments, an amino acid segment can have a length less than 1000 amino acid residues (e.g., less than 500, less than 400, less than 350, less than 300, less than 200, or less than 100 amino acid residues). In other embodiments, an amino acid segment can have a length from about 20 to about 200 amino acid residues (e.g., about 30 to about 180 amino acid residues, or about 40 to about 150 amino acid residues).

The amino acid segments of the ligand can be contiguous with the amino acid segments of the biotin-binding polypeptide or they can be separated by amino acids inserted as a structural spacer. A spacer segment can be one or more amino acids. The one or more amino acids can include amino acids that are the same or that are different. For example, a spacer can be a repeating series of a neutral amino acid (e.g., glycine, alanine, valine, isoleucine or leucine) ranging in number from 1 to 10 or more (e.g., 1, 2, 3, 4, 5, 6, 7, 8, 9, 10 or more). Another example of a spacer configuration can be a series of interspersed amino acids that may be neutral (e.g., glycine-alanine-glycine-alanine-glycine-alanine, or glycine-glycine-glycine-valine-valine-valine) or charged amino acids (e.g., glutamate-glutamate-glutamate-arginine-arginine-arginine, or aspartate-lysine-aspartate-lysine-aspartate-lysine) or amino acids with other functional groups (e.g., proline-proline-proline-serine-serine-serine or tyrosine-glutamine-cysteine-methionine-tryptophan) ranging in number from 1 to 10 or more (e.g., 1, 2, 3, 4, 5, 6, 7, 8, 9, 10 or more). In another embodiment, a spacer configuration can be a sequence of amino acids derived from a naturally occurring protein such as the hinge region joining the heavy chain CH1 and CH2 domains of immunoglobulin G.

A fusion protein can be produced in vitro by continuous peptide synthesis according to standard chemical methods know to those in the art. Synthetic polypeptides can also be purchased from commercial sources.

A fusion protein can also be produced by recombinant DNA techniques. Nucleic acid segments encoding the ligand can be operably linked in the same open reading frame to nucleic acid sequences encoding the biotin-binding polypeptide in a vector that includes the requisite regulatory elements, e.g., promoter sequences, transcription initiation sequences, and enhancer sequences, for expression in prokaryotic or eukaryotic cells. Methods well known to those skilled in the art can be used to construct expression vectors containing relevant coding sequences and appropriate transcriptional/translational control signals. Alternatively, suitable vector systems can be purchased from commercial sources.

Nucleic acid segments encoding ligands and biotin-binding polypeptides are available in the public domain. Examples of nucleic acid segments encoding ligands include, without limitation, the erythrocyte (glycophorin A)-binding antigen of Plasmodium falciparum EBA-175 (Bharara et al., Mol. Biochem. Parasitol. 138: 123-9, 2004), or mouse anti-human glycophorin A monoclonal antibody heavy chain (GenBank accession #AAZ67132) and corresponding light chain (Genbank accession #AAA21366)). Exemplary avidin and streptavidin sequences include those encoding CAC34569.1 (public GI:13397826) and AAN38297.1 (public GI:23507705) and GenBank accession numbers CAA00084.1 (public GI:14606); and ACL82594.1 (public GI:220900811), respectively.

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. The nucleic acid molecules can be synthesized (for example, by phosphoramidite based synthesis) or obtained from a biological cell, such as the cell of a mammal. The nucleic acids can be those of an animal, e.g., humans, a non-human primates, cattle, horses, pigs, sheep, goats, deer, elk, dogs, cats, mustelids, rabbits, guinea pigs, hamsters, rats, mice, fish or birds.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids disclosed herein also can be obtained by mutagenesis of, e.g., a naturally occurring DNA.

We use the terms “identity” and “identical” in connection with protein or DNA sequences to refer to the subunit sequence identity between two molecules. When a subunit position in both of the molecules is occupied by the same monomeric subunit (i.e., the same amino acid residue or nucleotide), then the molecules are identical at that position. Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence. As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence can be aligned to one or more subject nucleic acid or amino acid sequences using the computer program ClustalW (version 1.83, default parameters), which allows alignments of nucleic acid or protein sequences to be carried out across their entire length (global alignment). Chenna et al., Nucleic Acids Res., 31(13):3497-500 (2003). ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

Alternatively, percent sequence identity can be determined by comparing a target nucleic acid or amino acid sequence to the identified nucleic acid or amino acid sequence using the BLAST 2 Sequences (B12seq) program from the stand-alone version of BLASTZ containing BLASTN version 2.0.14 and BLASTP version 2.0.14. This stand-alone version of BLASTZ can be obtained on the World Wide Web from the U.S. government's National Center for Biotechnology Information web site (ncbi.nlm.nih.gov). Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -l; -r is set to 2; and all other options are left at their default setting. The following command will generate an output file containing a comparison between two sequences: C:\B12seq -i c:\seq1.txt -j c:\seq2.txt -p blastn -o c:\output.txt -q -l -r 2. If the target sequence shares homology with any portion of the identified sequence, the designated output file will present those regions of homology as aligned sequences. If the target sequence does not share homology with any portion of the identified sequence, the designated output file will not present aligned sequences.

Once aligned, a length is determined by counting the number of consecutive residues from the target sequence presented in alignment with sequence from the identified sequence starting with any matched position and ending with any other matched position. A matched position is any position where an identical residue is presented in both the target and identified sequence. Gaps presented in the target sequence are not counted since gaps are not amino acids or nucleotides. Likewise, gaps presented in the identified sequence are not counted since target sequence residues are counted, not residues from the identified sequence. The percent identity over a particular length is determined by counting the number of matched positions over that length and dividing that number by the length followed by multiplying the resulting value by 100.

It will be appreciated that different regions within a single amino acid or nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Where a mutant polypeptide differs from a reference sequence (e.g., a portion of a wild type immunoglobulin or (strept)avidin), the differences may constitute a substitution of one or more amino acid residues. The substitution can be, but is not necessarily, a “conservative” substitution. Examples of conservative substitutions include substitutions within the following groups: glycine, alanine; valine, isoleucine, leucine; aspartic acid, glutamic acid; asparagine, glutamine; serine, threonine; lysine, arginine; and phenylalanine, tyrosine.

Amino acid substitutions can be made, in some cases, by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine) Substitutions made within these groups can be considered conservative substitutions. Non-limiting examples of useful substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some embodiments, a polypeptide can include one or more non-conservative substitutions. Non-conservative substitutions typically entail exchanging a member of one of the classes described above for a member of another class. The substitutions may also be of an amino acid residue that does not occur in nature (e.g., a beta-amino acid (e.g., β alanine or norleucine). Polypeptides can also differ from a corresponding wild type sequence by virtue of the manner in which they are post-translationally modified (e.g., glycosylated).

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid.

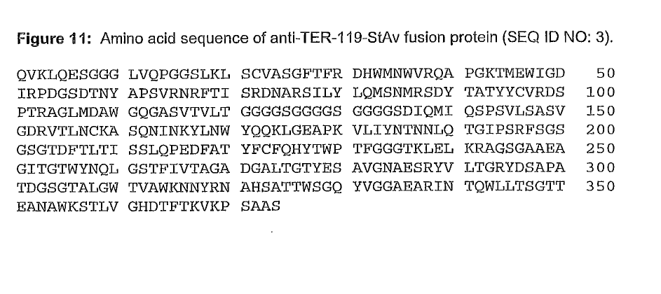

More specifically, the delivery proteins can include the amino acid sequence shown in FIG. 11 and having the sequence represented by SEQ ID NO: 3. The delivery proteins can be at least 80%, at least 85%, at least 87%, at least 90%, at least 95%, at least 97%, at least 98%, or at least 99% identical to the sequence represented by SEQ ID NO: 3. The mutation can consist of a change in the number or content of amino acid residues. For example, the mutant delivery protein can have a greater or a lesser number of amino acid residues than the sequence represented by SEQ ID NO: 3. Alternatively, or in addition, the mutant polypeptide can contain a substitution of one or more amino acid residues that are present in the sequence represented by SEQ ID NO: 3. The mutant polypeptide can differ from the sequence represented by SEQ ID NO: 3 by the addition, deletion, or substitution of a single amino acid residue.

By way of illustration, a polypeptide that includes an amino acid sequence that is at least 95% identical to a reference amino acid sequence of the sequence represented by SEQ ID NO: 3 is a polypeptide that includes a sequence that is identical to the reference sequence except for the inclusion of up to 23 alterations of the sequence represented by SEQ ID NO: 3. For example, up to 5% of the amino acid residues in the reference sequence may be deleted or substituted with another amino acid, or a number of amino acids up to 5% of the total amino acid residues in the reference sequence may be inserted into the reference sequence.

These alterations of the reference sequence may occur at the amino (N—) or carboxy (C—) terminal positions of the reference amino acid sequence or anywhere between those terminal positions, interspersed either individually among residues in the reference sequence or in one or more contiguous groups within the reference sequence.

A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

Vectors typically contain one or more regulatory regions. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

The vectors also can include, for example, origins of replication, scaffold attachment regions (SARs), and/or markers. A marker gene can confer a selectable phenotype, e.g., antibiotic resistance, on a cell. In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. A useful sequence for directing cellular localization is the pelB leader sequence, which can direct a protein to the periplasmic membrane of E. coli, where the sequence is removed by pelB peptidase. Sequences that direct periplasmic localization contribute to correct formation of disulfide bonds. Such tags can be inserted anywhere within the polypeptide, including at either the carboxyl or amino terminus. The choice of tag sequences and localization sequences will depend in part on the particular host cell selected for polypeptide production, e.g., a bacteria, yeast, mammalian or plant cell.

The expression vectors disclosed herein containing the above described coding can be used, for example, to transfect or transduce either prokaryotic (e.g., bacteria) cells or eukaryotic cells (e.g., yeast, insect, mammalian or plant) cells. Such cells can then be used, for example, for large or small scale in vitro production of the relevant fusion protein by methods known in the art. In essence, such methods involve culturing the cells under conditions which maximize production of the fusion protein and isolating the fusion protein from the cells or from the culture medium.

### Complexes.

In another embodiment, the ligand and biotin-binding protein can be obtained separately, either through chemical synthesis or synthesis in vivo, purified and then linked non-covalently or covalently. A useful non-covalent linkage is a biotin-avidin linkage. The term “biotin-avidin linkage” as used herein refers to any linkage via biotin or a biotin derivative or biomimic (e.g., Strep-Tag (IBA, St. Louis, Mo.)) and avidin or an avidin derivative, streptavidin, or biotin-binding fragments or subunits of avidin or streptavidin.

Thus, the biotinylated ligand can be linked to a biotin-binding polypeptide or fragment thereof. Methods for forming biotin-avidin linkages are well known to those in the art. (See for example, Handbook of Affinity Chromatography, (Chromatographic Sciences Series, vol. 63) ed. T. Kline, ISBN: 0824789393—Marcel Dekker (1993). (Strept)avidin and (Strept)avidin derivatives are available from commercial sources (Pierce Biotechnology, Rockford, Ill.; Invitrogen, Carlsbad, Calif.).

The ligand and the biotin-binding protein can also be synthesized as separate entities (by either chemical synthetic or recombinant methods) and then linked together by standard chemical methods known in the art. Chemical cross-linking agents can be homo-bifunctional (the same chemical reaction takes place at each end of the linker) or hetero-bifunctional (different chemical reactions take place at the ends of the linker). The chemistries available for such linking reactions include, but are not limited to, reactivity with sulfhydryl, amino, carboxyl, diol, aldehyde, ketone, or other reactive groups using electrophilic or nucleophilic chemistries, as well as photochemical cross-linkers using alkyl or aromatic azido or carbonyl radicals. An example of a targeted complex coupled via a homobifunctional cross-linking reagent can be a complex of an anti-band 3 monoclonal antibody as a red blood cell-targeting ligand and streptavidin as the biotin-binding protein linked by disuccinimidyl suberate (DSS, Pierce, Rockford, Ill.). An example of a delivery protein that includes a heterobifunctional cross-linking reagent can be an anti-glycophorin A monoclonal antibody as the ligand and streptavidin as the biotin-binding protein linked by N-succinimidyl 3-[2-pyridyldithio]-propionamido (SPDP). In this example, the antibody is first derivatized at sulfhydryl groups with SPDP's pyridyldithio reactivity, followed by the addition of streptavidin, whose amino residues react with SPDP's succinimidyl groups.

Examples of chemical cross-linking agents include, without limitation, glutaraldehyde, carbodiimides, bisdiazobenzidine, and N-maleimidobenzoyl-N-hydroxysuccinimide ester. Chemical cross-linkers are widely available from commercial sources (e.g., Pierce Biotechnology (Rockford, Ill.); Invitrogen (Carlsbad, Calif.); Sigma-Aldrich (St. Louis, Mo.); and US Biological (Swampscott, Mass.)).

In another embodiment, the ligand and the biotin-binding protein can be connected through a linking polymer. Examples of linking molecules include, but are not limited to linear or branched polymers or co-polymers (e.g., polyalkylene, poly(ethylene-lysine), polymethacrylate, polyamino acids, poly- or oligosaccharides, dendrimers). The ligand and the biotin-binding protein can be attached to the linking molecule or microparticle through a non-covalent high affinity linkage, e.g., streptavidin-biotin high affinity binding or chemical cross-linking techniques as described above.

For example, a polymer-supported delivery protein can be formed using a poly(ethylene-lysine) backbone. Such a linear copolymer backbone can be synthesized using bis(succinimidyl) poly(ethylene glycol₂₀₀₀) (Fluka Chemicals) to react with the α and ε amino groups of lysine. The available carboxyl termini of the lysines can be activated using (1-ethyl-3-[3-dimethylaminopropyl]carbodiimide Hydrochloride) (EDC, Pierce Biotechnology) in preparation for coupling amine-containing compounds. The total length of the co-polymer can be determined, in part, by the duration of the polymerization reaction. The targeting and IMG units can be combined in various ratios since there are up to 10 positions in a 2000 dalton co-polymer built with, e.g., PEG units of 2,000 dalton. Co-polymers using shorter PEG units (e.g., 500 daltons) can also be synthesized.

One example of a polymer-based delivery protein can be the addition of equimolar amounts of a ligand (e.g., an anti-glycophorin A monoclonal antibody) and biotin-binding protein (e.g., streptavidin) to produce a complex with a co-polymeric scaffold studded with targeting antibody molecules as well as biotin-binding molecules.

In another embodiment, the ligand and the biotin-binding protein can be connected through a microparticle. Examples of linking microparticles include, but are not limited to, micelles, liposomes, fullerenes, nanotubes, or other colloidal complexes such as lipoproteins. Liposomes and micelles can be prepared by methods described in Lasic D D, 1998, TIBTech 16:307. Fullerenes and nanotubes can be purchased from American Dye Source (www.adsdyes.com). Lipoproteins can be purchased from Biodesign International (www.biodesign.com).

The ligand and the biotin-binding protein can be attached to the linking molecule or microparticle through a non-covalent high affinity linkage, e.g., avidin-biotin high affinity binding or chemical cross-linking techniques as described above.

Alternatively or in addition, the ligand and/or the biotin-binding protein can be adsorbed or incorporated into a hydrophobic microparticle by hydrophobic affinity. A ligand and/or biotin-binding protein with an available hydrophobic domain can spontaneously associate with a hydrophobic microparticle by hydrophobic partitioning. The hydrophobic domain on the ligand and/or biotin-binding protein can be a polyamino acid stretch comprised of repeating or mixed hydrophobic amino acids (e.g., poly-Ala, poly-Gly, poly-Leu, poly-Ile, or Ala-Gly-Leu-Ile (SEQ ID NO: 5), etc.) or a bilayer-spanning polypeptide from a known trans-membrane protein, such as membrane IgM), alkyl chains (e.g., fatty acyl), or other hydrophobic structure (e.g., steroid). Such hydrophobic sequences can be naturally occurring sequences within the ligand and/or biotin-binding protein. Alternatively, such sequences can be introduced into the native amino acid sequence of the ligand or biotin-binding protein by standard recombinant DNA technology. The recombinant protein can be expressed and purified as described above.

The delivery proteins disclosed herein can include one or more of the same ligands or any combination of different ligands. The delivery proteins can also include one or more of the same biotin-binding protein or any combination of different biotin-binding proteins. Thus, the delivery proteins can include ligands that contain multiple copies (e.g., 2, 3, 4, 5, 6, or more) of a single ligand or a single copy of multiple (e.g., 2, 3, 4, 5, 6, or more) ligands or multiple copies (e.g., 2, 3, 4, 5, 6, or more) of two or more ligands (e.g., 2, 3, 4, 5, 6 or more).

### Methods of Use

The delivery proteins disclosed herein are generally useful for generating immune responses and as prophylactic or therapeutic vaccines or immune response-stimulating therapeutics. As used herein, “prophylaxis” can mean complete prevention of the symptoms of a disease, a delay in onset of the symptoms of a disease, or a lessening in the severity of subsequently developed disease symptoms. As used herein, “therapy” can mean a partial or complete abolishment of the symptoms of a disease or a decrease in the severity of the symptoms of the disease.

The delivery proteins can be used to target a payload, e.g., an immunogen, toxin or infectious agent, to the surface of a non-lymphoid cell for transport to the RES for degradation and presentation to the immune system. An immunogen can be a polypeptide, carbohydrate, glycolipid, hapten or biomimetic thereof. A polypeptide immunogen can include without limitation, a compound of two or more subunit amino acids, amino acid analogs, or other peptidomimetics, regardless of post-translational modification, e.g., biotinylation, phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

The amino acid sequence of the immunogens disclosed herein can be identical to the wild-type sequences of appropriate components. Alternatively, any of the components can contain mutations such as deletions, additions, or substitutions. All that is required is that the variant immunogen have at least 5% (e.g., 10%, 20%, 30%, 40%, 50%, 60%, 70%, 80%, 90%, 95%, 99%, 100%, or even more) of the ability of the immunogen containing only wild-type sequences to induce an immune response against the naturally occurring wild-type immunogen. Substitutions will preferably be conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

A polypeptide immunogen can include any peptide epitopes of a variety of lengths, for example, 7-50 (e.g., 7, 8, 9, 10, 11, 12, 13, 14, 15, 16, 17, 18, 19, 20, 21, 22, 23, 24, 25, 26, 27, 28, 29, 30, 32, 34, 36, 38, 40, 45, or 50) amino acid residues long. A polypeptide immunogen can include one or more epitopes, for example, 1, 2, 4, 6, 10, 20, 30, 50 or more.

Polypeptide immunogens can include one or more post-transcriptional modifications as described above. An immunogen can be, without limitation, biotinylated, glycosylated, acetylated, alkylated, isoprenylated, lipoylated, or phosphorylated.

An immunogen can also be a molecule that is not a protein, e.g., a small molecule such as nicotine, a carbohydrate or a glycolipid. Examples of carbohydrate and glycolipid immunogens, include, without limitation, bacterial lipopolysaccharide (LPS) or a fragment of it, microbial products bound by various Toll-like receptors (TLRs), bacterial diacyl and triacyl lipopeptides and lipoteichoic acid from bacteria, and zymosan from yeast cell walls.

The immunogen can be present in a killed or attenuated organism, in a crude cellular extract, a cell lysate or partially or substantially pure. The term “substantially pure” with respect to a naturally-occurring immunogen refers to an immunogen that has been separated from cellular components by which it is naturally accompanied, such that it is at least 60% (e.g., 70%, 80%, 90%, 95%, or 99%), by weight, free from naturally-occurring organic molecules with which it is naturally associated. Methods for purifying immunogens are known to those in the art. For example, in general, a substantially pure polypeptide will yield a single major band on a non-reducing polyacrylamide gel.

The immunogen can be a molecule expressed or released by any of a wide range of infectious agents, including, without limitation, viruses, viroids, bacteria, fungi, prions or parasites.

For example, viral pathogens can include, without limitation, influenza viruses, including the strain A(H1N5 or H1N1), hepatitis viruses (e.g., Hepatitis A, B, C and D), Arenaviruses, Bunyaviruses, Flaviviruses, Filoviruses, Alphaviruses, (e.g., Venezuelan equine encephalitis, eastern equine encephalitis, western equine encephalitis), Hantaviruses, human immunodeficiency viruses HIV1 and HIV2, feline immunodeficiency virus, simian immunodeficiency virus, measles virus, rabies virus, rotaviruses, papilloma virus, respiratory syncytial virus, Variola, and viral encephalitides, (e.g., West Nile Virus, LaCrosse, California encephalitis, VEE, EEE, WEE, Japanese Encephalitis Virus, Kyasanur Forest Virus). Bacterial pathogens can include, but are not limited to, Bacillus anthracis, Yersinia pestis, Yersinia enterocolitica, Clostridium botulinum, Clostridium perfringens Francisella tularensis, Brucella species, Salmonella spp., including Salmonella enteriditis, Escherichia coli, including E. coli O157:H7, Streptococcus pneumoniae, Staphylococcus aureus, Burkholderia mallei, Burkholderia pseudomallei, Chlamydia spp., Coxiella burnetii, Rickettsia prowazekii, Vibrio spp., Shigella spp. Listeria monocytogenes, Mycobacteria tuberculosis, M. leprae, Borrelia burgdorferi, Actinobacillus pleuropneumoniae, Helicobacter pylori, Neisseria meningitidis, Bordetella pertussis, Porphyromonas gingivalis, and Campylobacter jejuni.

Fungal pathogens can include, without limitation, members of the genera Aspergilllus, Penecillium, Stachybotrys, Trichoderma, mycoplasma, Histoplasma capsulatum, Cryptococcus neoformans, Chlamydia trachomatis, and Candida albicans.

Pathogenic protozoa can include, for example, members of the genera Cryptosporidium, e.g., Cryptosporidium parvum, Giardia lamblia, Microsporidia and Toxoplasma, e.g., Toxoplasma brucei, Toxoplasma gondii, Entamoeba histolytica, Plasmodium falciparum, Leishmania major and Cyclospora cayatanensis.

Examples of useful immunogens derived from pathogenic organisms include, for example, but are not limited to, influenza A M2 protein, hepatitis B surface antigen, HBV preS1 protein, HIV tat, HIV gp120, anthrax protective antigen, and botulinum toxin. An influenza M2 protein antigen can be the ectodomain peptide M2e, for example, SLLTEVETPIRNEWGCRCNDSSD (SEQ ID NO: 6), or a variant of the ectodomain peptide M2e, for example, SEQ ID NO: 7 or SEQ ID 8. An HBV preS1 protein can include the preS1 protein peptide 35-49, e.g., FGANSNNPDWDFNPNKDHWPEANQVGA (SEQ ID NO: 9). Examples of useful non-peptidic immunogens include the pneumococcal polysaccharides from Streptococcus pneumoniae.

The immunogen can also be a molecule expressed by an animal. For example, an immunogen can be a molecule whose expression is correlated with a particular disease state, for example, cancer, autoimmune or neurodegenerative disease.

Thus, the immunogen can be a tumor-associated antigen (TAA). As used herein, a TAA is a molecule (e.g., a polypeptide, carbohydrate or lipid) that is expressed by a tumor cell and either (a) differs qualitatively from its counterpart expressed in normal cells, or (b) is expressed at a higher level in tumor cells than in normal cells. Thus, a TAA can differ (e.g., by one or more amino acid residues where the molecule is a protein) from, or it can be identical to, its counterpart expressed in normal cells. Preferably it is not expressed by normal cells. Alternatively, it is expressed at a level at least two-fold higher (e.g., a two-fold, three-fold, five-fold, ten-fold, 20-fold, 40-fold, 100-fold, 500-fold, 1,000-fold, 5,000-fold, or 15,000-fold higher) in a tumor cell than in the tumor cell's normal counterpart. Examples of relevant cancers include, without limitation, hematological cancers such as leukemias and lymphomas, neurological tumors such as astrocytomas or glioblastomas, melanoma, breast cancer, lung cancer, head and neck cancer, gastrointestinal tumors such as gastric or colon cancer, liver cancer, pancreatic cancer, genitourinary tumors such ovarian cancer, vaginal cancer, bladder cancer, testicular cancer, prostate cancer or penile cancer, bone tumors, and vascular tumors. Relevant TAAs include, without limitation, carcinoembryonic antigen (CEA), RAGE, MART (melanoma antigen), MAGE (melanoma antigen) 1-4, 6 and 12, MUC (mucin) (e.g., MUC-1, MUC-2, etc.), tyrosinase, Pmel 17 (gp100), GnT-V intron V sequence (N-acetylglucoaminyltransferase V intron V sequence), Prostate cancer psm, PRAME (melanoma antigen), β-catenin, MUM-1-B (melanoma ubiquitous mutated gene product), GAGE (melanoma antigen)1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP) Bc1-2, prostate specific antigen (PSA), and Ki-67.

Other useful immunogens include those derived from antigens that are involved in the initiation or progression of neurodegenerative diseases, e.g., Alzheimer's disease and Transmissible Spongiform Encephalopathies (TSEs), e.g., human prion diseases such as Creutzfeld-Jacob disease (CJD), variant CJD (“mad cow disease'), Gerstmann-Straussler-Scheinker syndrome (GSS); Fatal familial Insomnia (FFI); animal prion diseases such as Scrapie in sheep; bovine spongiform encephalopathy (BSE) in cows; transmissible mink encephalopathy (TME) in mink; chronic wasting disease (CWD) in elk and deer.

As used herein, a “neurodegenerative antigen” is a molecule (e.g., a polypeptide, carbohydrate or lipid) that is expressed by a neuronal cell in an individual with a neurodegenerative disease and either (a) differs qualitatively from its counterpart expressed in cells from an individual who does not have the neurodegenerative disease, e.g., the molecule appears in abnormal locations within the body or is associated with other molecules not normally found with the antigen in healthy individuals who do not have the neurodegenerative disease, or (b) is expressed at a higher level in cells from an individual who does not have the neurodegenerative disease. Thus, a neurodegenerative antigen can differ (e.g., by one or more amino acid residues where the molecule is a protein) from, or it can be identical to, its counterpart expressed in normal cells. It is preferably not expressed by normal cells. Alternatively, it is expressed at a level at least two-fold higher (e.g., a two-fold, three-fold, five-fold, ten-fold, 20-fold, 40-fold, 100-fold, 500-fold, 1,000-fold, 5,000-fold, or 15,000-fold higher) in a tumor cell than in the tumor cell's normal counterpart.

Examples of neurodegenerative antigens found in Alzheimer's disease include beta-amyloid, tau protein, alpha synuclein. Other neurodegenerative disease antigens can be derived from prions. As defined herein, a prion is small proteinaceous infectious particle that resists inactivation by procedures that modify nucleic acids. Prions are encoded by the prion-related protein gene (PrP). Mutant forms of the PrP protein aggregate as prions which can lead to fatal neurodegenerative disease. Thus, an immunogen can be a PrP polypeptide.

Germ cell immunogens can be useful in the generation of immune responses that block the function of germ cells, thereby interfering with conception. Germ cell antigens can include antigens on sperm cells. Examples include, without limitation sperm adhesion molecule 1 (SPAM-1), and human intra-acrosomal protein.

An immunogen can also be a non-toxic variant of a toxic substance (a “toxoid”) or of a microorganism such as a bacterium (a “bacterin”) that can be used to stimulate an immune response against the harmful form of the toxin. A toxoid can be, without limitation, a toxin that has been rendered less toxic or completely non-toxic through treatment with high temperature, aggregation, chemical reaction (e.g., formalin fixation), coupling to a carrier molecule, or molecular alteration (e.g., deletion, augmentation or substitution). A toxoid can be thus derived from a toxin such as, for example, ricin, anthrax or botulinum toxin types A, B, C, D, E, F or G. A bacterin can be, without limitation, a bacterial preparation that has been rendered less toxic or completely non-toxic through treatment with high temperature, aggregation, chemical reaction (e.g., formalin fixation), coupling to a carrier molecule, or molecular alteration (e.g., deletion, augmentation or substitution). A bacterin can thus be derived from a pathogenic bacterium such as, for example, bovine salmonella or swine E. Coli.

An immunogen can also be a substance of abuse such as nicotine, or an opiate or opiate derivative. Such an immunogen can induce antibodies capable of binding and neutralizing the corresponding substance of abuse.

The delivery protein and the payload can be administered as a complex. For example, the payload can be modified by the addition of one or more biotin molecules and then linked to the biotin binding moiety of the delivery protein via a (strept)avidin-biotin linkage or a an anti-biotin-biotin linkage. The term “biotin-avidin linkage” as used herein refers to any linkage via biotin or a biotin derivative or biomimic (e.g., Strep-Tag (IBA, St. Louis, Mo.)) and avidin or an avidin derivative, streptavidin, or biotin-binding fragments or subunits of avidin or streptavidin.

Thus, the delivery protein can be linked to a biotinylated immunogen via (strept)avidin or biotin-binding fragments or subunits of (strept)avidin. Generally, when (strept)avidin and biotin-bearing molecules are combined, they link spontaneously and firmly. Methods for forming biotin-avidin linkages are well known to those in the art. (See for example, Handbook of Affinity Chromatography, (Chromatographic Sciences Series, vol. 63) ed. T. Kline, ISBN: 0824789393—Marcel Dekker (1993), which is herein incorporated by reference. Assembly of the delivery protein: payload heterocomplexes can be performed in any order. The composition of the assembled delivery protein: payload heterocomplexes can be validated by standard methods known in the art, for example, SDS-PAGE and western blotting and LC/MS methods.

Alternatively, the components of the heterocomplex, i.e., the delivery protein and the payload can be administered separately, either simultaneously or in sequence so that the heterocomplex is formed in vivo. The components can be administered in any order, for example, the delivery protein can be administered, followed by a biotinylated immunogen or the immunogen can be administered followed by the delivery protein.

The delivery protein and the payload can also be linked through a biotin-streptavidin linkage that includes an additional biotinylated immunoglobulin. For example, a delivery protein can be linked to a biotinylated antibody that specifically binds the immunogen. By varying the relative ratios of the ligand and the biotin-binding polypeptide and/or the relative affinity of the biotin-binding polypeptide used in the delivery protein, it is possible to generate delivery proteins with various ratios of delivery protein: payload. Thus, delivery protein: payload heterocomplexes can be prepared to include one molecule of the delivery protein and three molecules of the payload; two molecules each of delivery protein and immunogen; or three molecules of delivery protein and one molecule of immunogen. Such variant heterocomplexes, e.g., single and double biotin-binding complexes may be useful for modulating the binding of the heterocomplexes to circulating non-lymphoid cells, e.g., red blood cells.

Alternatively, a polynucleotide containing a nucleic acid sequence encoding a delivery protein of interest can be delivered to an appropriate cell of the animal. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm).

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression.

In the relevant polynucleotides (e.g., expression vectors) the nucleic acid sequence encoding the delivery protein of interest with an initiator methionine and optionally a targeting sequence is operatively linked to a promoter or enhancer-promoter combination. Promoters and enhancers are described above.

As described for the polypeptides above, a nucleic acid encoding a delivery protein can be administered simultaneously or coordinately with a payload, e.g., a biotinylated immunogen, toxin or infectious agent. Alternatively, the subject can be treated with a nucleic acid encoding a delivery protein and before or after the administration of a payload, e.g., a biotinylated immunogen, toxin or infectious agent. In another embodiment, a subject could be treated with a nucleic acid encoding a biosynthetically biotinylated immunogen or a biomimic amino acid sequence followed either prior to or subsequent to administration of a delivery protein or a nucleic acid encoding such a delivery protein. Biosynthetic biotinylation can be accomplished by incorporating a substrate sequence that results in biotinylation of a lysine residue within said sequence by biotin protein ligase (Chapman-Smith A, Cronan J E (1999) Biomolec. Engineer. 16:119-125). Alternately, it is possible to insert a nucleotide sequence coding for the peptide, PCHPQFPRCYA, which acts as a biotin biomimic and has the ability to bind streptavidin (Hu W G, Alvi A Z, Fulton R E, Suresh M R, Nagata L P (2002) Hybrid Hybridomics. 21:415-20). Such methods will result in the expression of a streptavidin-binding immunogen protein in vivo in the host.

Polynucleotides can be administered in a pharmaceutically acceptable carrier. Pharmaceutically acceptable carriers are biologically compatible vehicles which are suitable for administration to a human or other subject, e.g., physiological saline. A therapeutically effective amount is an amount of the polynucleotide which is capable of producing a medically desirable result (e.g., a T cell response) in a treated subject. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

The methods disclosed herein can be applied to a wide range of species, e.g., humans, non-human primates (e.g., monkeys), horses, cattle, pigs, sheep, deer, elk, goats, dogs, cats, mustelids, rabbits, guinea pigs, hamsters, rats, mice, fish, for example salmon, carp and tilapia, and birds, for example, chickens, turkeys, ducks, geese and pigeons. Thus, they can be used, for example, as vaccines or therapeutic agents against infectious diseases, including diseases that can potentially result from bioterrorism attacks. The heterocomplexes can be used in the preparation of a medicament for treatment of an infectious disease. Infectious diseases can include diseases caused by any of the pathogens listed herein. Examples include, without limitation, influenza, HIV-AIDS, hepatitis, botulism, plague, smallpox, tularemia, viral hemorrhagic fevers, brucellosis, gastrointestinal disease induced by pathogenic forms of E. coli, Salmonella and Shigella, glanders, melioidosis, psittacosis, Q fever, Staph infection, typhus fever, viral encephalitis, water and foodborne safety threats, cholera, diphtheria, endocarditis, Legionaire's disease, Listeriosis, periodontal disease, Asperigillosis, Blastomycosis, histoplasmosius, trypanosomiasis, malaria, Giardiasis, Schistosomiasis, toxoplasmosis, smallpox, west Nile virus.

The heterocomplexes are useful therapeutics and prophylactics for a wide variety of conditions. The heterocomplexes can be useful as both prophylactics and therapeutics for cancer (e.g., any of those recited above). The heterocomplexes can be employed to stimulate an immune response against cells in a cancer patient or can be administered in cases where a subject is at relatively high risk for a cancer (e.g., lung cancer in a tobacco smoker or melanoma in a subject with multiple nevi). Moreover, as described above, the heterocomplexes can also be useful in therapy or prophylaxis of neurodegenerative diseases. Thus the heterocomplexes can be administered to an individual with Alzheimer's disease or transmissible spongiform encephalopathies (TSEs, also known as prion diseases) administered to an individual who is at risk for developing Alzheimer's disease or a TSE. The heterocomplexes are applicable to various autoimmune conditions, e.g., rheumatoid arthritis or psoriasis and can be used to stimulate an immune response against a pro-inflammatory protein, e.g., TNF-α. The heterocomplexes disclosed herein can also be useful as a contraceptive vaccine, when the immunogen is a germ cell antigen or human chorionic gonadotropin.

The heterocomplexes disclosed herein can also be useful as prophylactics and post-exposure therapeutics against medical conditions that result from exposure to toxins. Such targeted immune conjugate complexes that include non-toxic variants of toxic substances, e.g., ricin, botulinum toxin, nicotine and other drugs, or non-toxic variants of pathogenic bacteria, e.g., salmonella and E. coli, can be used to stimulate an immune response against the harmful form of the toxin, and thus protect against or mitigate the potential damage the toxin or drug may cause.

The heterocomplexes can be administered directly to a subject, e.g., a human or an animal. The heterocomplexes can be used in the preparation of a medicament. Generally, the heterocomplexes can be suspended in a pharmaceutically-acceptable carrier (e.g., physiological saline). A composition can be made by combining any of the heterocomplexes provided herein with a pharmaceutically acceptable carrier. Such carriers can include, without limitation, or exclude, sterile aqueous or non-aqueous solutions, suspensions, and emulsions. Examples of non-aqueous solvents include mineral oil, propylene glycol, polyethylene glycol, vegetable oils, and injectable organic esters, for example. Aqueous carriers include, without limitation, or exclude water, alcohol, saline, and buffered solutions. Preservatives, flavorings, and other additives such as, for example, antimicrobials, anti-oxidants, chelating agents, inert gases, and the like also may be present. It will be appreciated that any material described herein that is to be administered to a subject can contain one or more pharmaceutically acceptable carriers.

Any composition described herein can be administered to any part of the host's body. A composition can be delivered to, without limitation, the joints, nasal mucosa, blood, lungs, intestines, muscle tissues, skin, or peritoneal cavity of a subject. In addition, a composition can be administered by intravenous, intraperitoneal, intramuscular, subcutaneous, intramuscular, intrarectal, intravaginal, intrathecal, intratracheal, intradermal, or transdermal injection, by oral or nasal administration, by inhalation, or by gradual perfusion over time. In a further example, an aerosol preparation of a composition can be given to a host by inhalation.

The dosage required depends on the route of administration, the nature of the formulation, the nature of the patient's illness, the subject's size, weight, surface area, age, and sex, other drugs being administered, and the judgment of the attending physician. Suitable dosages are in the range of 0.01-1,000 μg/kg. Wide variations in the needed dosage are to be expected in view of the variety of heterocomplexes available and the differing efficiencies of various routes of administration. Variations in these dosage levels can be adjusted using standard empirical routines for optimization as is well understood in the art. Administrations can be single or multiple (e.g., 2- or 3-, 4-, 6-, 8-, 10-, 20-, 50-, 100-, 150-, or more fold). Encapsulation of the heterocomplexes in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a heterocomplex can be administered once a month for three months or once a year for a period of ten years. It is also noted that the frequency of treatment can be variable. For example, a heterocomplex can be administered once (or twice, three times, etc.) daily, weekly, monthly, or yearly.

Alternatively or in addition the heterocomplexes can be administered along with an adjuvant. An “adjuvant” is any agent that can enhance an immune response against a particular antigen such as a polypeptide. Examples of adjuvants include alum and other aluminum-based compounds (e.g., Al₂O₃). Aluminum-based compounds can be obtained from various commercial suppliers. Other adjuvants include immuno-stimulating complexes (ISCOMs) that can contain such components as cholesterol and saponins; one or more additional immunostimulatory components, including, without limitation, muramyldipeptide (e.g., N-acetylmuramyl-L-alanyl-D-isoglutamine; MDP), monophosphoryl-lipid A (MPL), and formyl-methionine containing tripeptides such as N-formyl-Met-Leu-Phe. Such compounds are commercially available from Sigma Chemical Co. (St. Louis, Mo.), for example. Other adjuvants can include CpG oligodeoxynucleotides (Coley Pharmaceuticals), QS21 (Cambridge Biotech) and MF59 (Chiron).

Substances that act as adjuvants that stimulate cells of the RES can be co-administered with the heterocomplexes described herein and result in augmented immunization. Such substances include but are not limited to granulocyte monocyte colony-stimulating factor (GM-CSF) and interferon gamma (IFNγ). Such RES-stimulatory substances (“cytokines”) can be co-administered with the IMG-containing heterocomplexes as physical admixtures. In another embodiment, the RES-stimulatory substances can be biotinylated so that both the IMG and the cytokine are jointly targeted to the RBC surface for transport to RES cells.

The compositions provided herein can contain any ratio of adjuvant to heterocomplex. The adjuvant: heterocomplex ratio can be 50:50 (vol:vol), for example. Alternatively, the adjuvant: heterocomplex ratio can be, without limitation, 90:10, 80:20, 70:30, 64:36, 60:40, 55:45, 40:60, 30:70, 20:80, or 90:10.

An effective amount of any composition provided herein can be administered to a host. The term “effective” as used herein refers to any amount that induces a desired immune response while not inducing significant toxicity in the host. Such an amount can be determined by assessing a host's immune response after administration of a known amount of a particular composition. In addition, the level of toxicity, if any, can be determined by assessing a host's clinical symptoms before and after administering a known amount of a particular composition. It is noted that the effective amount of a particular composition administered to a host can be adjusted according to a desired outcome as well as the host's response and level of toxicity. Significant toxicity can vary for each particular host and depends on multiple factors including, without limitation, the host's disease state, age, and tolerance to pain.

Any method can be used to determine if a particular immune response is induced. For example, antibody responses against a particular immunogen can be determined using an immunological assay (e.g., ELISA or lymphocyte proliferation assay). In such an assay, the wells of a microtiter plate can be coated with the immunogen and incubated with serum from a subject treated with the heterocomplex designed to produce antibodies against the corresponding immunogen in that subject, and the presence or absence of antibodies against the immunogen can be determined by standard methods know to those in the art. In addition, clinical methods that can assess the degree of a particular disease state can be used to determine if a desired immune response is induced. For example, in a cancer patient, a reduction in tumor burden can indicate a desired immune response in a patient treated with a composition designed to stimulate an immune response against a tumor antigen expressed on the patient's tumor.

The heterocomplexes provided herein can be administered in conjunction with other therapeutic modalities to an individual in need of therapy. The heterocomplexes can be given prior to, simultaneously with or after treatment with other agents. In the case of infectious disease, the heterocomplexes can be administered in conjunction with any antimicrobial agent, e.g., an antibiotic, e.g., including, without limitation, aminoglycosides, cephalosporins, macrolides, penicillins, peptides, quinolones, sulfonamides, tetracyclines; an antiviral, including without limitation, amantadine, rimantadine, zanamavir and oseltamivir; an anti-fungal, including, without limitation, echinocandin, caspofungin, anidulafungin; or anti-parasitic agent, including, without limitation, chlorquine, mebendazole, and clotrimazole.

The heterocomplexes can also be used in conjunction with standard anti-cancer therapies, including, without limitation, chemotherapy, e.g., alkylating agents, anthracyclines, cycloskeletal disruptors, topoisomerase inhibitors, nucleotide analogues, platinum-based agents, retinoids, vinca alkaloids; radiation therapy, hormone ablation and surgery. The heterocomplexes can also be used in conjunction with other therapeutics for neurodegenerative diseases, including donepezil, galantamine, memantine.

### Articles of Manufacture

Also disclosed are articles of manufacture that can include the delivery proteins as provided herein. Components and methods for producing articles of manufacture are well known. An article of manufacture can include, for example, one or more delivery proteins. In addition, an article of manufacture further may include, for example, packaging materials, instructions for use, buffers or other control reagents for treating or monitoring the condition for which prophylaxis or treatment is required. In some embodiments, an article of manufacture may include one or more adjuvants.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Materials and Methods

### Plasmid Construction.

A synthetic plasmid (Blue Heron Biotechnology) was designed to encode the C-terminal one-third of the Ter scFv (TerC). The TerC sequence in this plasmid started with the Hind III site within the published DNA sequence. The stop codon was replaced with Bam HI and XhoI sites to allow additional sequences to be cloned behind TerC without termination of the reading frame.

A plasmid encoding core streptavidin was digested with BamHI. The resulting 393 bp fragment, encoding core streptavidin, was ligated into the BamHI site of the plasmid described above, between the TerC and the XhoI sequences.

The resulting plasmid was digested with Hind III and Xho, releasing the TerC-streptavidin fragment. This was ligated in pET 21a(+) which had been digested with the same enzymes. As a result, the His-tag that already existed in pET 21a(+) was now appended in-frame to the 3′ end of the streptavidin sequence.

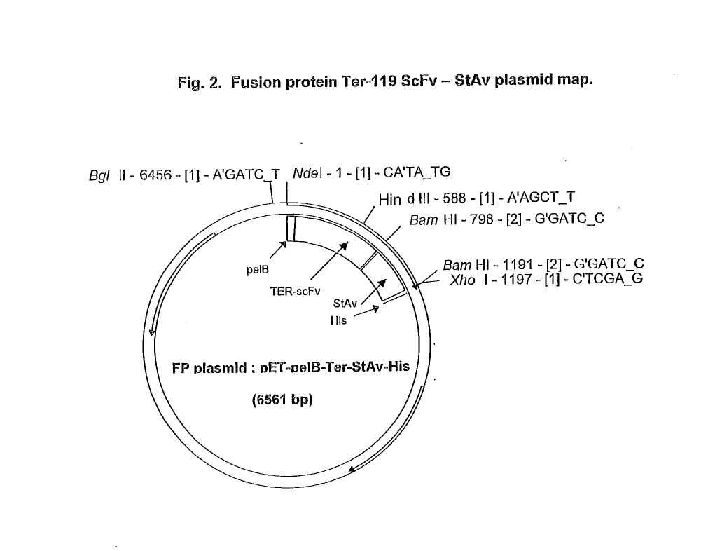

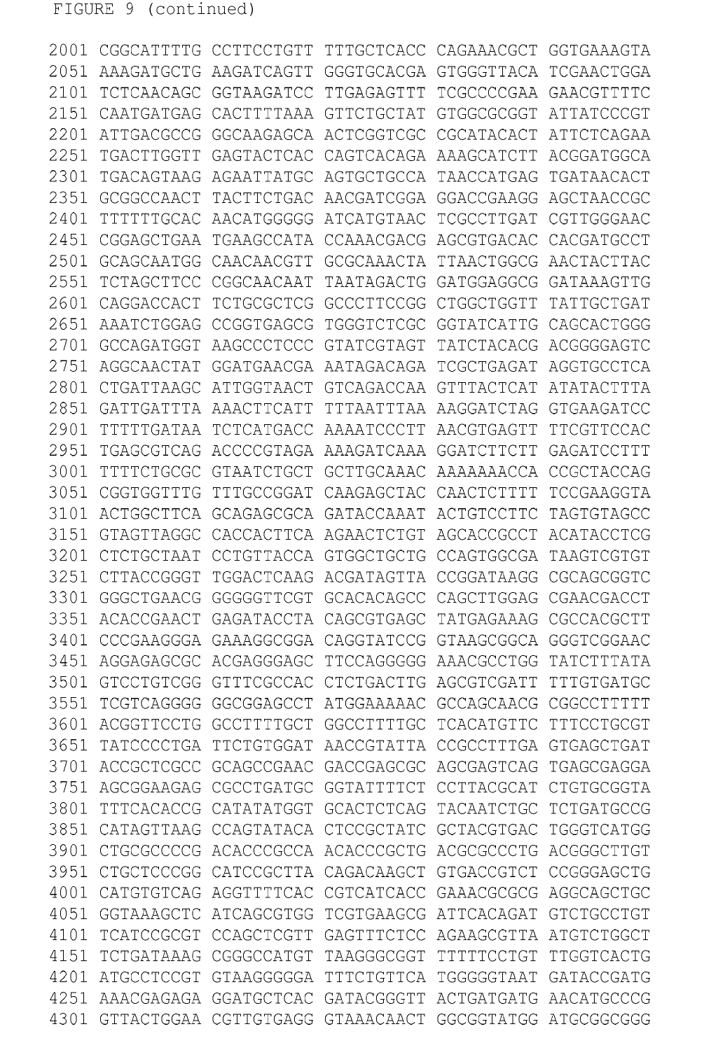

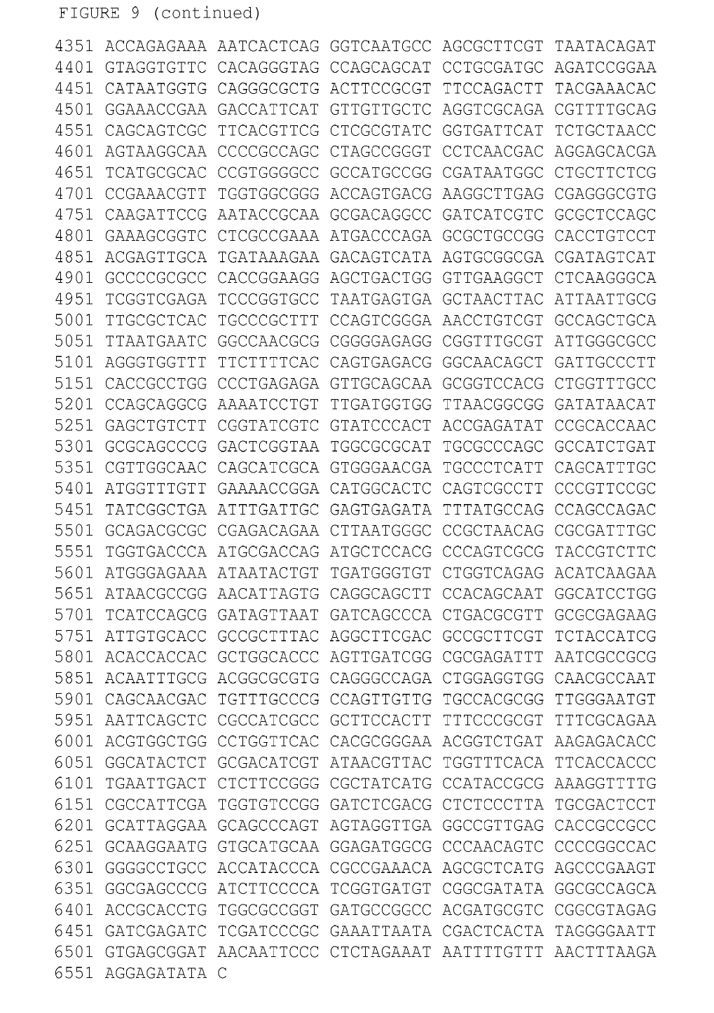

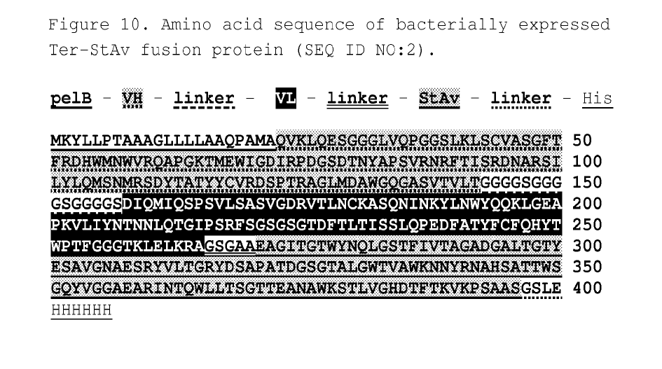

Another synthetic plasmid (Blue Heron Biotechnology) was designed to encode an NdeI site, pelB leader, and then the amino-terminal two-thirds of Ter (TerN, up to the Hind III site). This plasmid was digested with Nde I+Hind III. The released pelB-TerN fragment was ligated into the previous plasmid (pET 21a(+) encoding TerC-streptavidin-His), which had been digested with the same enzymes. The resulting pET-based plasmid, designated pelB-Ter-streptavidin-His FP, encoded pelB-Ter (full-length)-streptavidin-His. A map of pelB-Ter-streptavidin-His FP is shown in FIG. 2. The nucleotide sequence of the anti-TER-119-streptavidin fusion protein encoded by pelB-Ter-streptavidin-His construct is represented by the sequence extending from residue 69-1196 of SEQ ID NO:1 and is shown in FIG. 9. The amino acid sequence of the bacterially expressed pelB-Ter-streptavidin-His (SEQ ID NO: 2) is shown in FIG. 10. The amino acid sequence of the TER-119-streptavidin fusion protein (SEQ ID NO: 3) is shown in FIG. 11.

### M2e ELISA.

Microtiter ELISA plates (Immulon 1B, Thermo Scientific) were coated with M2e peptide. Peptide was dissolved to 50 ng/mL in PBS containing 1 mM DTT and 100 μL was pipetted into each well. Plates were incubated at ambient temperature overnight and the wells were aspirated. Plates were washed with PBS containing 0.05% Tween-20 (PBST). Wells were blocked with 200 μL PBS containing 1% BSA (PBSB) for 2 h and washed twice with 200 μL PBST. Test sera (or the anti-M2e monoclonal antibody 14C2) were added in 100 μL using for diluent PBSB containing 1% normal mouse serum (Balb/c strain), and incubated at ambient temperature for 2.5 h, followed by 3 washes of 200 μL PBST. Goat anti-mouse IgG conjugated with horseradish peroxidase (Sigma), diluted 1:1,000 in PBSB, was added at 100 μL/well and incubated for 1 h. Plates were washed 4 times with PBST. TMB substrate (Thermo Scientific, 100 μL) was added to the wells for 20 min at ambient temperature in the dark. The color reaction was stopped by the addition of 100 μL 1 M HCl and absorbance was read at A₄₅₀-A₅₆₀. For the purpose of logarithmic plotting of the titers, samples with no measurable anti-M2e IgG were assigned the value of 0.01 μg/mL.

### Streptavidin ELISA.

The streptavidin ELISA method was similar to that of the M2e ELISA, except that the plates were coated with streptavidin (Sigma) and the reference anti-streptavidin monoclonal antibody was S10D4 (Abcam).

### Example 2

### Production of Fusion Protein

The FP plasmid, pelB-Ter-streptavidin-His FP, was constructed as described in Example 1. The construct included the pelB leader sequence for periplasmic expression, but because there was indication that the leader sequence was cleaved, whole cell lysates were used for FP recovery.

### Fusion Protein Expression.

The pelB-Ter-streptavidin-His FP was expressed in E. coli BL21 (DE3) pLysS cells. For expression, cells were grown in shake flasks at 37° C. in LB to an OD600 of 1, then induced with 1 mM IPTG. After incubation for another 3 h at 37° C., the cells were pelleted for 20 min at 5,000×G at 2° C. Cell pellets were stored at −20° C. until purification.

### Fusion Protein Purification.

Cell pellets from 700 mL of culture were suspended in 20 mL of buffer WB (1 M urea/1% Triton X-100/50 mM Tris-HCl, pH 7.5) and poured into a capped 50 mL tube. The tube was inverted and vortexed periodically for about 15 minutes at room temperature, during which time the viscosity gradually decreased. MgCl₂ (36 μl of a 1 M stock) and benzonase (320 units) were then added to further decrease the viscosity. After another 15 min at room temperature, the volume was raised to 36 mL with additional WB. The lysate was then subjected to centrifugation at 12,000×G for 20 min at 20° C. The FP pelleted under these conditions. The pellet was washed with 36 mL of WB, and the FP was pelleted as before.

The washed pellet was suspended in 10 mL of 8 M urea/60 mM Tris-HCl, pH 8.0 and allowed to dissolve with occasional mixing for 2.5 hours at 4° C. The suspension was then subjected to centrifugation at 5000×G for 3 minutes at room temperature. The supernatant, which contained the FP, was then passed over a column containing His-Select resin (Sigma) which had been equilibrated with 8 M urea/60 mM Tris-HCl, pH 8.0. The column was washed with the same buffer. The FP was eluted in the same buffer after supplementation with 100 mM imidazole.

For refolding, the protein was first diluted in 8 M urea/60 mM Tris-HCl, pH 8.0 to 200 μg/ml. The diluted protein was then dialyzed sequentially against 4 M and then 2 M urea in 40 mM Tris-HCl/0.4 M arginine/1 mM DTT, pH 7.5. The protein was further dialyzed against 1.5 M and then 1 M urea in the same buffer, except lacking DTT. The protein was then further dialyzed against 5 mM NaPi/70 mM NaCl/400 mM arginine-HCl, pH 7.4 (PBSR).

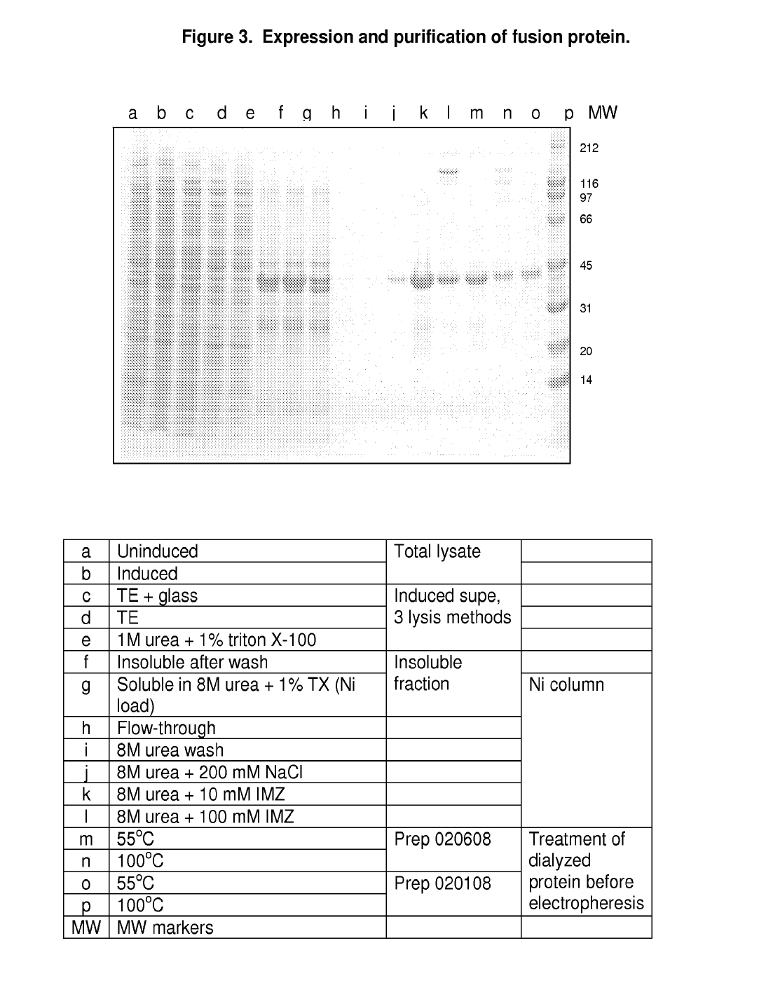

Purification of the FP was monitored by SDS polyacrylamide gel analysis as shown in FIG. 3. To determine whether the refolded protein had formed tetramers (as does native streptavidin), aliquots were heated slightly (40° C.) in sample buffer and subjected to electrophoresis on reducing SDS gels. As shown in FIG. 3, lanes m and o), about one-third of the refolded FP had an electrophoretic mobility that was consistent with that of a tetrameric form.

### Example 3

### Binding of FP to Murine Red Blood Cells (mRBCs) In Vitro

Binding of FP to mRBC In Vitro.

FP binding to murine red blood cells (mRBCs) in vitro was examined in cell ELISA experiments using fresh mRBC drawn by retroorbital bleeding with heparinized capillary tubes from Balb/c female mice. All steps in the binding assays were conducted on ice. Blood was washed 3 times in PBS and the pellet was resuspended in PBS containing 1% BSA (PBSB). Aliquots of 10⁶ cells were dispensed into 1.5 mL microcentrifuge tubes previously blocked with PBSB. FP at the indicated concentrations was added to the cells, which were incubated at 4° C. for 45 min. Unbound FP was removed by 3 washes in PBSB. mRBC-bound FP was detected by the addition of 4 μg biotinylated β-galactosidase (Sigma), at approximately 5-fold molar excess over the added FP, for 45 min. After washing the cells 3 times to remove unbound galactosidase, the substrate ONPG (Sigma) was prepared according to the vendor's instructions to 6.8 mg/mL and 100 μL was added to each tube and color development proceeded at ambient temperature. Color was recorded at 405 nm using a microtiter plate reader (Multiskan MCC 341).

Detection of TER-119 on mRBCs.

The presence of TER-119 on the surface of mRBCs was detected in similar cell ELISA conditions. Erythrocytes were incubated with 1 μg of anti-mouse TER-119, a rat IgG2b monoclonal antibody (eBioscience), followed by the addition of goat anti-rat IgG conjugated with β-galactosidase (Southern Biotech) at a dilution of 1:500. ONPG was used as the substrate. Rat IgG2b (eBioscience) was used as isotype control.

FP bound to mRBC in a dose-dependent manner (Table 1). Binding of FP was shown to be specific for surface TER-119 in a competition binding experiment. Washed mRBC were treated with 2.5 μg anti-TER-119 monoclonal antibody for 45 min, after which FP was added (without washing the cells). After washing the cells, the binding of FP was measured as described above. Pre-treatment of the mRBC with anti-TER-119 Mab inhibited FP binding by approximately 50% when compared to pre-treatment with isotype control rat IgG2b (Table 1).

TABLE 1
Binding of FP to Mouse RBCs In Vitro

| | | | Binding | % |
|---|---|---|---|---|
| FP (lot) | FP (μg) | Competitor | (OD units) | inhibition |
| 052308 | 0.29 | — | 214 | |
| | 1.45 | — | 360 | |
| | 7.25 | — | 1203 | |
| 080408 | 0.5 | — | 25 | |
| | 0.5 | control IgG | 17 | |
| | 0.5 | anti-TER | 34 | 0 |
| | 3.0 | — | 121 | |
| | 3.0 | control IgG | 83 | |
| | 3.0 | anti-TER | 47 | 61 |
| | 9.0 | — | 202 | |
| | 9.0 | control IgG | 249 | |
| | 9.0 | anti-TER | 147 | 27 |

### Example 4

### Coupling of M2e Peptide to FP

M2e Peptide.

The M2e peptide (NH₂-SLLTEVETPIRNEWGCRCNDSSD-COOH) (SEQ ID NO: 6) was chemically synthesized (Bio-World, Dublin, Ohio). Biotinylated peptide was synthesized by adding a biotinylated lysine residue to the C-terminal Asp (D).

### Formation of Immune Complexes.

Biotinylated M2e peptide was dissolved in 100 mM NH₄CO₃ (pH 7.0). An aliquot (83 μg) was then mixed with dialyzed FP (1.1 mg) in a reaction volume of 7.2 mL. The reaction buffer was PBS containing 0.4 M arginine (PBSR). After incubation for 20 min at room temperature, the mixture was passed over polymyxin B-agarose resin (Sigma), then passed through a sterile syringe filter (Pall, part number 4602) that had been pre-blocked with 10% normal mouse serum and then rinsed with PBSR. Endotoxin levels were below the limit of detection (5 EU/ml, Lancaster Labs). The complexes were stored at −20° C. until ready for injection into mice.

### Analysis of Immune Complexes.

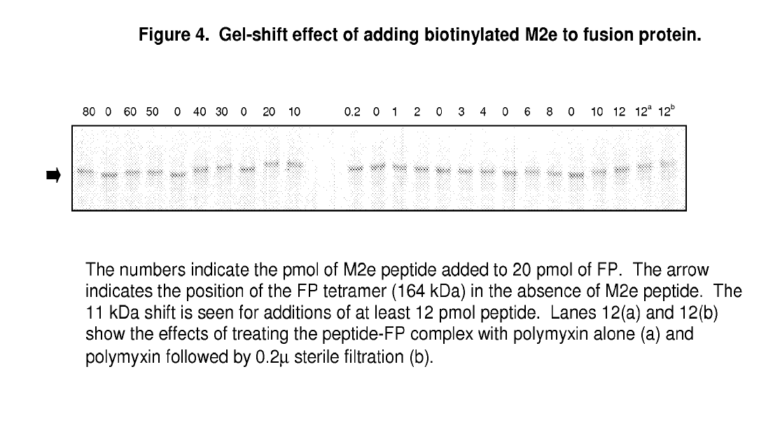

Gel-shift assays were used to determine the amount of biotinylated M2e peptide required to alter the mobility of FP in reducing SDS gels. Serial dilutions of biotinylated M2e peptide at 0.2 to 80 pmol were mixed with 20 pmol FP preparation and the migration of FP was monitored. Results shown in FIG. 4 indicated that 10-12 pmole peptide were required to fully shift the mobility of 20 pmole FP. Only the 164 kDa band, which represented approximately 30% of the total protein, exhibited altered migration, suggesting that only the tetrameric form of FP actively bound biotinylated peptide. Accordingly, complexes of peptide and FP used for immunization were prepared at a molar ratio of 1 mol M2e peptide:1 mol FP (monomer).

### Example 5

### Immunogenicity of M2e-FP Complexes

### Immunization of Mice.

Mice were used under the protocol guidelines of the University of Delaware Institutional Animal Care and Use Committee. Female Balb/c mice (25 g) were injected intravenously (IV) in the tail vein with a volume of 0.2 mL, except for inocula containing alum, which were injected in the same volume subcutaneously (SC).

Free M2e peptide was inoculated at doses of 30, 10 and 3.3 μg. M2e peptide adsorbed onto alum was inoculated at doses of 30 and 0.33 μg. M2e peptide (biotinylated at the C-terminus) was also coupled with the FP for final inoculation doses of M2e of 0.33, 0.11 and 0.037 μg. All inocula were dissolved in PBSR. Mice were given a primary inoculum on day 0 and they were boosted on days 14 and 28. Blood was collected 7 days after each inoculation (and on day −2 for baseline) and serum was stored frozen.

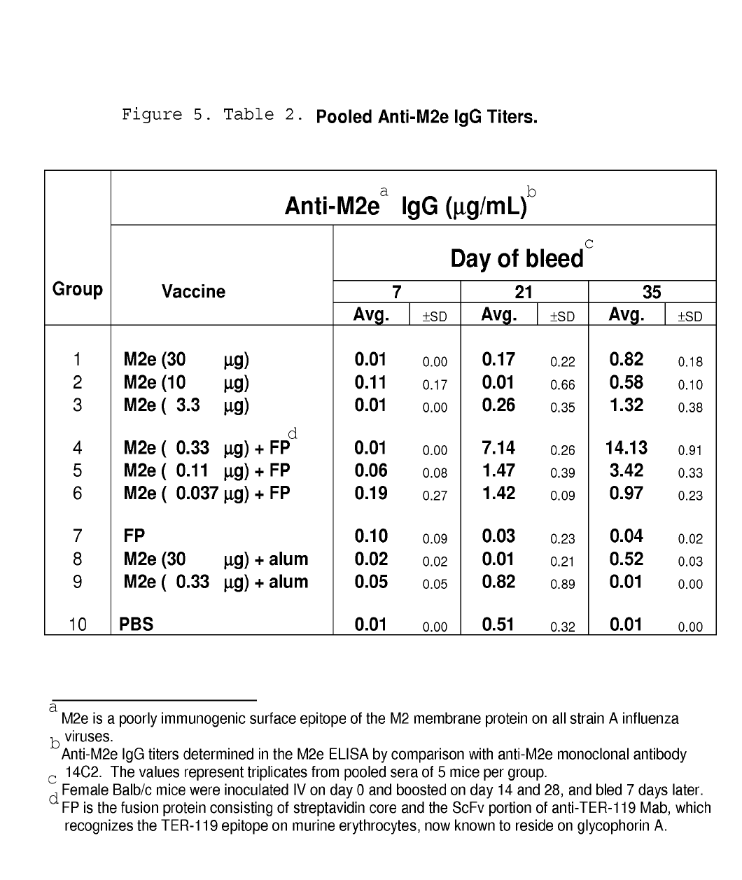

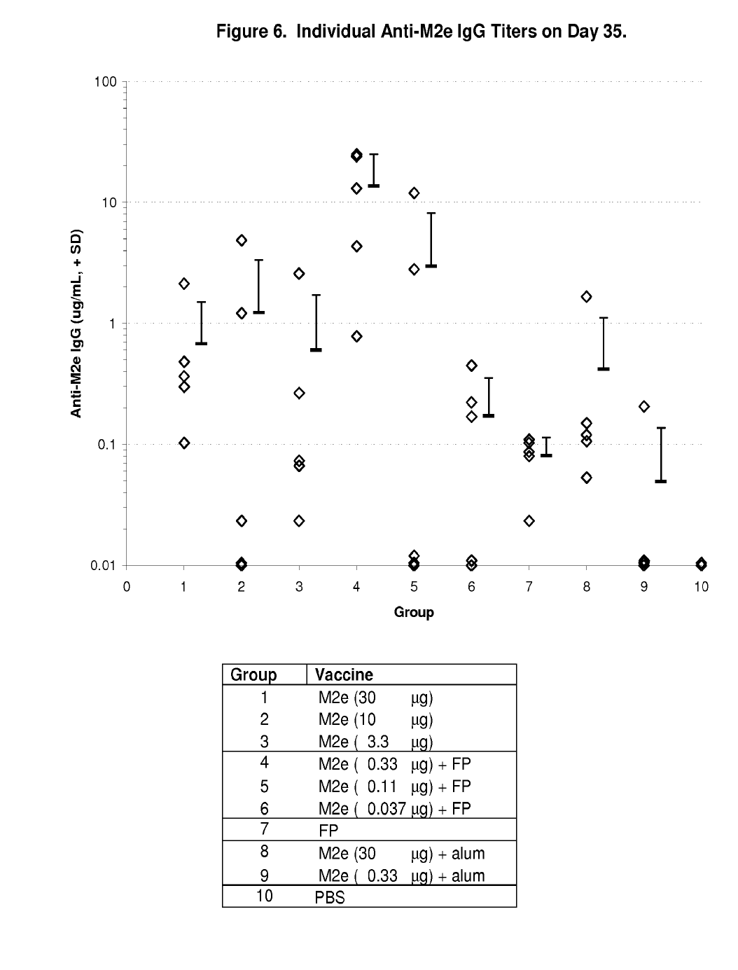

Mice were immunized in groups of five. Both pooled and individual sera were analyzed by ELISA according to the methods described in Example 1. The results from the analysis of pooled sera, shown in FIG. 5, indicated that mice inoculated with M2e peptide coupled to FP elicited a substantial immune response (Table 2). There was a dose-response relationship, with the highest dose of coupled peptide (0.33 μg) giving a titer of 7.14 μg/mL anti-M2e IgG by day 21 (7 days after the first boost), which rose further to 14.13 μg/mL. Even the lowest dose of 0.037 μg peptide elicited an IgG titer, 1.42 μg/mL by day 21 and 0.97 μg/mL on day 35. Peptide alone or peptide adsorbed onto alum generated a weaker IgG response even at the highest dose of 30 μg peptide (less than 1 μg/mL). Analysis of individual sera on day 35 are presented in FIG. 6. These results confirmed the low immunogenicity of M2e peptide alone (or in combination with alum). Taken together, the data in FIGS. 5 and 6 indicated that targeting the M2e peptide to the red blood cell surface with the immunotargeting FP construct resulted in a potently immunogenic preparation. The potency increase is a minimum of 800-fold (30 μg/0.037 μg). Considering that only about 30% of the FP preparation appeared to bind peptide (see Example 4, above), the data suggested that the coupled peptide preparation is a minimum of 2,400-fold more potent than peptide alone.

### Example 6

### Immunogenicity of M2e-FP Complexes: Comparison of Injection Routes

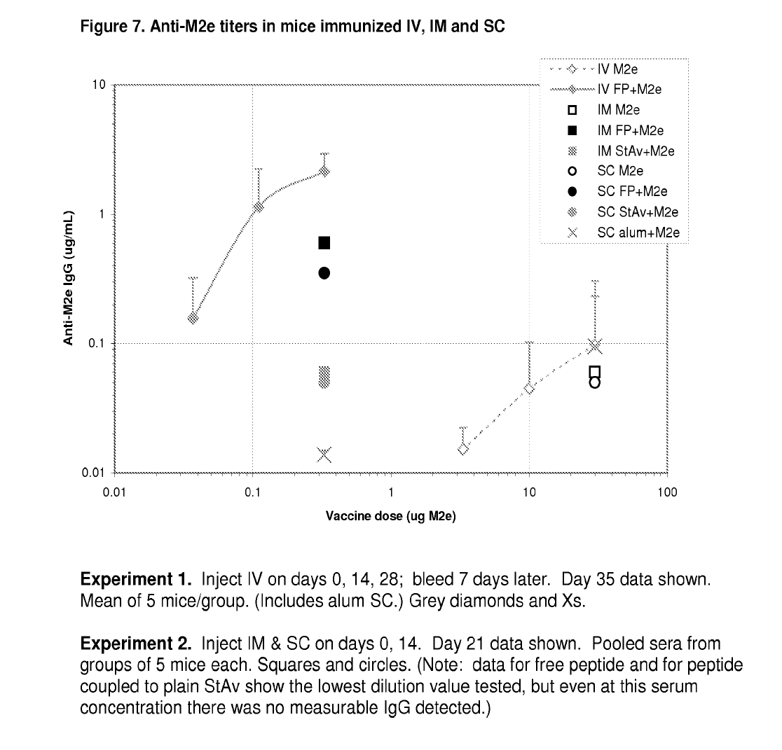

Injection routes and the requirement for the antibody fragment (scFv) in the FP were examined. Groups of 5 mice were used and pooled sera from each group were analyzed by ELISA as described in Example 1. In this experiment, however, only single doses of M2e peptide were used: for peptide alone a dose of 30 μg was used and for the combination with FP, the peptide dose was 0.33 μg. The results are summarized in FIG. 7 and they are superimposed on the data from Example 5 for comparison. Just as in the experiment of Example 5 using IV dosing, peptide coupled to FP elicited an IgG response at least one order of magnitude higher than free peptide, and did so at two orders of magnitude lower dose, for a total of three orders of magnitude augmented immunogenicity.

### Example 7

### Immunogenicity of M2e-FP Complexes: Anti-Streptavidin Titers

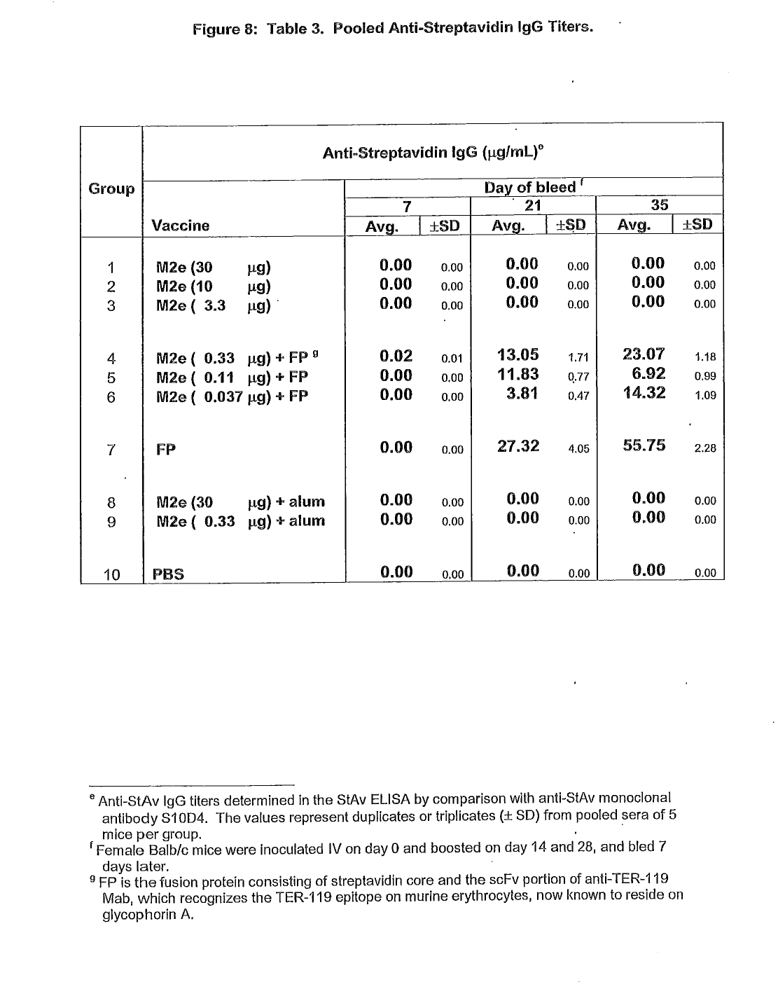

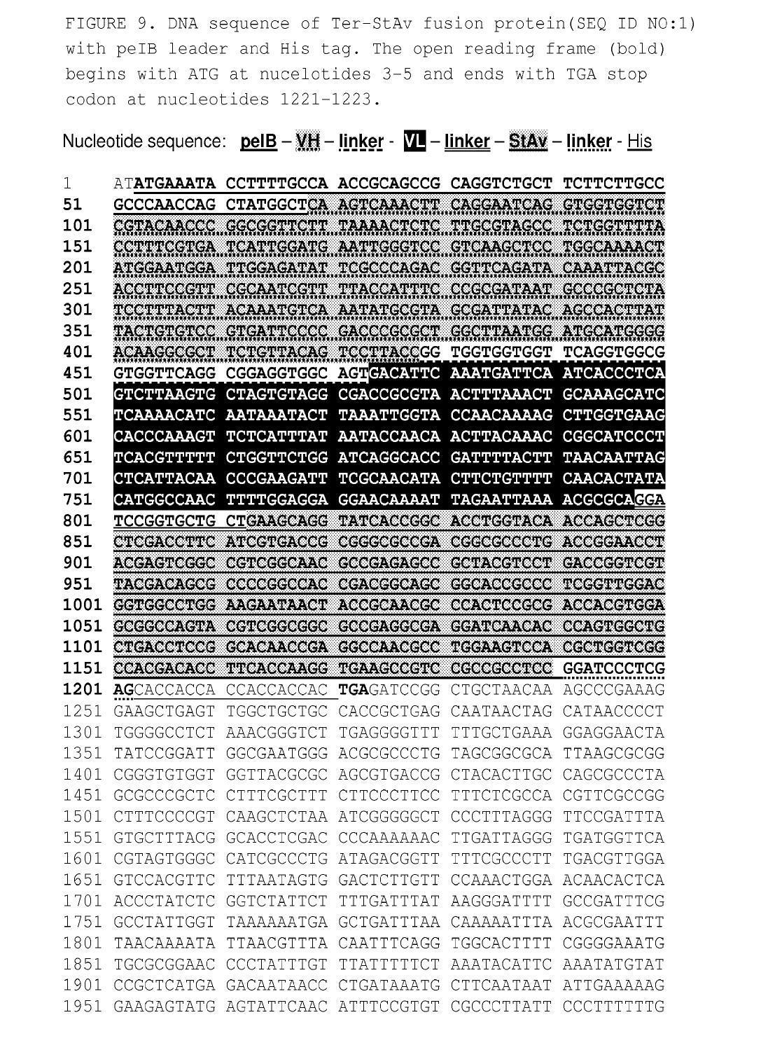

Pooled sera were also evaluated for their content of anti-streptavidin titers using the anti-streptavidin ELISA described in Example 1. The results shown in FIG. 8 indicated that streptavidin was immunogenic in mice and that an anti-streptavidin IgG response was generated. This anti-streptavidin response, however, did not impede the induction of anti-M2e immunity in either the primary response, when the anti-streptavidin titer was low, or in the secondary responses, when the titers were elevated.

A number of embodiments of the invention have been described. Nevertheless, it will be understood that various modifications may be made without departing from the spirit and scope of the invention. Accordingly, other embodiments are within the scope of the following claims. Publications cited herein and the material for which they are cited are hereby specifically incorporated by reference in their entireties.

1. A composition comprising a ligand that specifically binds to a cell surface molecule on a circulating non-lymphoid cell, wherein the cell-surface molecule is not CR1, joined to a biotin-binding protein or fragment thereof.

2. The composition of claim 1, wherein said cell surface molecule is selected from the group consisting of glycophorin A, band 3, Ter-119, blood group antigen H, blood group antigen A, blood group antigen B, CD41a, CD14, CD56, CD66d, CD83, CMKLR1, and BDCA-4.

3. The composition of claim 2, wherein the ligand is an antibody or a fragment thereof.

4. The composition of claim 3, wherein the antibody is an anti-TER-119 antibody, an anti-glycophorin A antibody, an anti-band 3 antibody, an anti-blood group antigen A antibody, an anti-blood group antigen B antibody, an anti-blood group antigen H antibody, an anti-CD41a antibody, an anti-CD 14 antibody, an anti-CD56 antibody, an anti-CD66d antibody, an anti-CD83 antibody, an anti-CMKLR1 antibody, or an anti-BDCA-4 antibody.

5. The composition of claim 1, wherein the antibody is a single chain antibody.

6. (canceled)

7. The composition of claim 1, wherein the cell-surface molecule is on a red blood cell.

8-9. (canceled)

10. The composition of claim 1, wherein the biotin-binding protein is streptavidin, avidin, neutravidin or an anti-biotin antibody.

11. (canceled)

12. The composition of claim 10, wherein the streptavidin comprises a core streptavidin.

13. The composition of claim 12, wherein the core streptavidin comprises amino acids 249 to 374 of SEQ ID NO: 3.

14. The composition of claim 1, wherein the ligand is joined to the biotin-binding protein by a covalent bond.

15. The composition of claim 14, wherein the ligand and the biotin-binding protein constitute a fusion protein.

16. The composition of claim 15, wherein the fusion protein comprises an anti-glycophorin A antibody and a core streptavidin.

17. The composition of claim 16, wherein the fusion protein comprises an amino acid sequence that is at least 80% identical to the amino acid sequence represented by SEQ ID NO: 3.

18-21. (canceled)

22. The composition of claim 16, wherein the fusion protein comprises the amino acid sequence represented by SEQ ID NO: 3.

23. The composition of claim 16, wherein the fusion protein consists of the amino acid sequence represented by SEQ ID NO: 3.

24. A nucleic acid sequence encoding the fusion protein of claim 16.

25. An expression vector comprising the nucleic acid sequence of claim 24.

26-28. (canceled)

29. A method for inducing or enhancing an immune response to an immunogen in a subject, the method comprising:
(a) providing a biotinylated immunogen;
(b) combining the immunogen of (a) with a composition consisting essentially of a ligand that specifically binds to a cell surface molecule on a circulating non-lymphoid cell, wherein the cell-surface molecule is not CR1, joined to a biotin-binding protein or fragment thereof, to form an immune complex; and
(c) administering an effective amount of the complex to the individual, wherein the complex induces or enhances an immune response to the immunogen.

30. The method of claim 29, wherein the immunogen is influenza A M2 protein or a fragment of influenza A M2 protein.

31-32. (canceled)

33. An article of manufacture comprising a measured amount of a delivery protein, wherein the delivery protein consists essentially of a ligand that specifically binds to a cell surface molecule on a circulating non-lymphoid cell, wherein the cell-surface molecule is not CR1, joined to a biotin-binding protein or fragment thereof, and one or more items selected from the group consisting of packaging material, a package insert comprising instructions for use, a sterile fluid, and a sterile container.

34-36. (canceled)


SEQUENCE LISTING (16 sequences)

SEQ ID NO: 1 - DNA, 6561 bp, Artificial Sequence
  Description of Artificial Sequence: Synthetic polynucleotide
atatgaaataccttttgccaaccgcagccgcaggtctgcttcttcttgccgcccaaccagctatggctcaagtcaaacttcaggaatcaggtggtggtctcgtacaacccggcggttctttaaaactctcttgcgtagcctctggttttacctttcgtgatcattggatgaattgggtccgtcaagctcctggcaaaactatggaatggattggagatattcgcccagacggttcagatacaaattacgcaccttccgttcgcaatcgttttaccatttcccgcgataatgcccgctctatcctttacttacaaatgtcaaatatgcgtagcgattatacagccacttattactgtgtccgtgattccccgacccgcgctggcttaatggatgcatggggacaaggcgcttctgttacagtccttaccggtggtggtggttcaggtggcggtggttcaggcggaggtggcagtgacattcaaatgattcaatcaccctcagtcttaagtgctagtgtaggcgaccgcgtaactttaaactgcaaagcatctcaaaacatcaataaatacttaaattggtaccaacaaaagcttggtgaagcacccaaagttctcatttataataccaacaacttacaaaccggcatcccttcacgtttttctggttctggatcaggcaccgattttactttaacaattagctcattacaacccgaagatttcgcaacatacttctgttttcaacactatacatggccaacttttggaggaggaacaaaattagaattaaaacgcgcaggatccggtgctgctgaagcaggtatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcacaaccgaggccaacgcctggaagtccacgctggtcggccacgacaccttcaccaaggtgaagccgtccgccgcctccggatccctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatac

SEQ ID NO: 2 - protein, 406 aa, Artificial Sequence
  Description of Artificial Sequence: Synthetic polypeptide
MKYLLPTAAAGLLLLAAQPAMAQVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSGGGGSGGGGSDIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKRAGSGAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASGSLEHHHHHH

SEQ ID NO: 3 - protein, 374 aa, Artificial Sequence
  Description of Artificial Sequence: Synthetic polypeptide
QVKLQESGGGLVQPGGSLKLSCVASGFTFRDHWMNWVRQAPGKTMEWIGDIRPDGSDTNYAPSVRNRFTISRDNARSILYLQMSNMRSDYTATYYCVRDSPTRAGLMDAWGQGASVTVLTGGGGSGGGGSGGGGSDIQMIQSPSVLSASVGDRVTLNCKASQNINKYLNWYQQKLGEAPKVLIYNTNNLQTGIPSRFSGSGSGTDFTLTISSLQPEDFATYFCFQHYTWPTFGGGTKLELKRAGSGAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 4 - skipped

SEQ ID NO: 5 - protein, 4 aa, Artificial Sequence
  Description of Artificial Sequence: Synthetic peptide
AGLI

SEQ ID NO: 6 - protein, 23 aa, Influenza A virus
SLLTEVETPIRNEWGCRCNDSSD

SEQ ID NO: 7-8 - skipped (2 entries)

SEQ ID NO: 9 - protein, 27 aa, Hepatitis B virus
FGANSNNPDWDFNPNKDHWPEANQVGA

SEQ ID NO: 10 - protein, 6 aa, Artificial Sequence
  Description of Artificial Sequence: Synthetic peptide
GAGAGA

SEQ ID NO: 11 - protein, 6 aa, Artificial Sequence
  Description of Artificial Sequence: Synthetic peptide
GGGVVV

SEQ ID NO: 12 - protein, 6 aa, Artificial Sequence
  Description of Artificial Sequence: Synthetic peptide
EEERRR

SEQ ID NO: 13 - protein, 6 aa, Artificial Sequence
  Description of Artificial Sequence: Synthetic peptide
DKDKDK

SEQ ID NO: 14 - protein, 6 aa, Artificial Sequence
  Description of Artificial Sequence: Synthetic peptide
PPPSSS

SEQ ID NO: 15 - protein, 5 aa, Artificial Sequence
  Description of Artificial Sequence: Synthetic peptide
YQCMW

SEQ ID NO: 16 - protein, 11 aa, Artificial Sequence
  Description of Artificial Sequence: Synthetic peptide
PCHPQFPRCYA